SEQUENCE LISTING (35 sequences)

SEQ ID NO: 1 - DNA, 48 bp, synthetic construct
  misc_feature 1..8: note = DNA
  misc_feature 9..17: note = RNA
  misc_feature 18..32: note = DNA
  misc_feature 33..41: note = RNA
  misc_feature 42..48: note = DNA
  source 1..48: mol_type = other DNA
gggaatccaaggaggcaggattcccgtcacagaaggaggcactgtgac

SEQ ID NO: 2 - DNA, 48 bp, synthetic construct
  misc_feature 1..8: note = DNA
  misc_feature 9..17: note = RNA
  misc_feature 18..32: note = DNA
  misc_feature 33..41: note = RNA
  misc_feature 42..48: note = DNA
  source 1..48: mol_type = other DNA
gggaacgtaagcctccaacgttcccggatgctaagcctccaagcatcc

SEQ ID NO: 3 - DNA, 78 bp, synthetic construct
  misc_feature 1..15: note = DNA
  source 1..78: mol_type = other DNA
  misc_feature 16..24: note = RNA
  misc_feature 25..54: note = DNA
  misc_feature 55..63: note = RNA
  misc_feature 64..78: note = DNA
gggaaaggaaggactaaggaggcaagtccttcctttccccgcgtatagtcacagaaggaggcactgtgactatacgcg

SEQ ID NO: 4 - DNA, 78 bp, synthetic construct
  misc_feature 1..15: note = DNA
  source 1..78: mol_type = other DNA
  misc_feature 16..24: note = RNA
  misc_feature 25..54: note = DNA
  misc_feature 55..63: note = RNA
  misc_feature 64..78: note = DNA
gggaaggaaggaagaaagcctccatcttccttccttccccgatgctctactaggaagcctccacctagtagagcatcg

SEQ ID NO: 5 - DNA, 34 bp, synthetic construct
  misc_feature 1..4: note = DNA
  source 1..34: mol_type = other DNA
  misc_feature 5..13: note = RNA
  misc_feature 14..21: note = DNA
  misc_feature 22..30: note = RNA
  misc_feature 31..34: note = DNA
gggaaaggaggcatcccgcccaaggaggcagggc

SEQ ID NO: 6 - DNA, 34 bp, synthetic construct
  misc_feature 1..3: note = DNA
  source 1..34: mol_type = other DNA
  misc_feature 4..12: note = RNA
  misc_feature 13..20: note = DNA
  misc_feature 21..29: note = RNA
  misc_feature 30..34: note = DNA
cgcaagcctccagcgttcgcaagcctccagcgaa

SEQ ID NO: 7 - RNA, 48 nt, synthetic construct
  source 1..48: mol_type = other RNA
gggaatccaaggaggcaggattcccgtcacagaaggaggcactgtgac

SEQ ID NO: 8 - RNA, 48 nt, synthetic construct
  source 1..48: mol_type = other RNA
gggaacgtaagcctccaacgttcccggatgctaagcctccaagcatcc

SEQ ID NO: 9 - DNA, 62 bp, synthetic construct
  misc_feature 1..15: note = DNA
  misc_feature 16..24: note = RNA
  misc_feature 25..46: note = DNA
  misc_feature 47..55: note = RNA
  misc_feature 56..62: note = DNA
  source 1..62: mol_type = other DNA
gagggaaatcccttcaaggaggcagaagggatttccctcgtcacagaaggaggcactgtgac

SEQ ID NO: 10 - RNA, 78 nt, synthetic construct
  source 1..78: mol_type = other RNA
gggaacgtaagcctccaacgttcccggatgctaagcctccaagcatccttcgtaaaaggacatatgagaccttcaatt

SEQ ID NO: 11 - RNA, 26 nt, synthetic construct
  source 1..26: mol_type = other RNA
ttgaaggtctcatatgtccttttacg

SEQ ID NO: 12 - DNA, 20 bp, synthetic construct
  source 1..20: mol_type = other DNA
ccttgaagggatttccctcc

SEQ ID NO: 13 - DNA, 20 bp, synthetic construct
  source 1..20: mol_type = other DNA
ggagggaaatcccttcaagg

SEQ ID NO: 14 - DNA, 74 bp, synthetic construct
  misc_feature 1..8: note = DNA
  misc_feature 9..17: note = RNA
  misc_feature 18..32: note = DNA
  misc_feature 33..41: note = RNA
  misc_feature 42..74: note = DNA
  source 1..74: mol_type = other DNA
gggaatccaaggaggcaggattcccgtcacagaaggaggcactgtgactatccgtccccccccccccacggata

SEQ ID NO: 15 - DNA, 74 bp, synthetic construct
  misc_feature 1..8: note = DNA
  misc_feature 9..17: note = RNA
  misc_feature 18..32: note = DNA
  misc_feature 33..41: note = RNA
  misc_feature 42..74: note = DNA
  source 1..74: mol_type = other DNA
gggaacgtaagcctccaacgttcccggatgctaagcctccaagcatcctatccgtccccccccccccacggata

SEQ ID NO: 16 - DNA, 52 bp, synthetic construct
  source 1..52: mol_type = other DNA
tatccgtccccccccccccacggatatatccgtccccccccccccacggata

SEQ ID NO: 17 - DNA, 74 bp, synthetic construct
  misc_feature 1..8: note = DNA
  misc_feature 9..17: note = RNA
  misc_feature 18..58: note = DNA
  misc_feature 59..67: note = RNA
  misc_feature 68..74: note = DNA
  source 1..74: mol_type = other DNA
gggaacgtaagcctccaacgttccccgcctaggttgggtagggtggtggcgggatgctaagcctccaagcatcc

SEQ ID NO: 18 - RNA, 77 nt, synthetic construct
  source 1..77: mol_type = other RNA
gggaatccaaggaggcaggattcccgtcacagaaggaggcactgtgactttggtggtgcagatgaacttcagggtca

SEQ ID NO: 19 - RNA, 25 nt, synthetic construct
  source 1..25: mol_type = other RNA
accctgaagttcatctgcaccaccg

SEQ ID NO: 20 - RNA, 27 nt, synthetic construct
  source 1..27: mol_type = other RNA
cggtggtgcagatgaacttcagggtca

SEQ ID NO: 21 - DNA, 15 bp, synthetic construct
  source 1..15: mol_type = other DNA
ggattcccgtcacag

SEQ ID NO: 22 - DNA, 15 bp, synthetic construct
  source 1..15: mol_type = other DNA
acgttcccggatgct

SEQ ID NO: 23 - DNA, 15 bp, synthetic construct
  source 1..15: mol_type = other DNA
gagggaaatcccttc

SEQ ID NO: 24 - DNA, 22 bp, synthetic construct
  source 1..22: mol_type = other DNA
gaagggatttccctcgtcacag

SEQ ID NO: 25 - DNA, 33 bp, synthetic construct
  source 1..33: mol_type = other DNA
ctgtgactatccgtccccccccccccacggata

SEQ ID NO: 26 - DNA, 33 bp, synthetic construct
  source 1..33: mol_type = other DNA
agcatcctatccgtccccccccccccacggata

SEQ ID NO: 27 - DNA, 26 bp, synthetic construct
  source 1..26: mol_type = other DNA
cgcctaggttgggtagggtggtggcg

SEQ ID NO: 28 - DNA, 15 bp, synthetic construct
  source 1..15: mol_type = other DNA
gggaaaggaaggact

SEQ ID NO: 29 - DNA, 30 bp, synthetic construct
  source 1..30: mol_type = other DNA
agtccttcctttccccgcgtatagtcacag

SEQ ID NO: 30 - DNA, 15 bp, synthetic construct
  source 1..15: mol_type = other DNA
ctgtgactatacgcg

SEQ ID NO: 31 - DNA, 15 bp, synthetic construct
  source 1..15: mol_type = other DNA
gggaaggaaggaaga

SEQ ID NO: 32 - DNA, 30 bp, synthetic construct
  source 1..30: mol_type = other DNA
tcttccttccttccccgatgctctactagg

SEQ ID NO: 33 - DNA, 15 bp, synthetic construct
  source 1..15: mol_type = other DNA
cctagtagagcatcg

SEQ ID NO: 34 - DNA, 11 bp, synthetic construct
  source 1..11: mol_type = other DNA
agggatttccc

SEQ ID NO: 35 - DNA, 11 bp, synthetic construct
  source 1..11: mol_type = other DNA
gggaaatccct

(19) United States
(12) Patent Application Publication
(10) Pub. No.: US 2025/0388621 A1
(43) Pub. Date: Dec. 25, 2025

(54) RNA-DNA FUSOMERS AND METHODS OF USE THEREOF

(71) Applicants: The University of North Carolina at Charlotte, Charlotte, NC (US); The Board of Trustees of the University of Illinois, Urbana, IL (US); Northeastern University, Boston, MA (US)

(72) Inventors: Kirill A. Afonin, Charlotte, NC (US); Yasmine Radwan, Charlotte, NC (US); Aleksei Aksimentiev, Urbana, IL (US); Meni Wanunu, Sharon, MA (US)

(21) Appl. No.: 19/189,926

(22) Filed: Apr. 25, 2025

Related U.S. Application Data
(60) Provisional application No. 63/663,827, filed on Jun. 25, 2024.

(51) Int. Cl. C07H 21/04; A61K 31/711; A61K 33/38; A61K 47/54; C12N 15/113; C12N 15/115

(52) U.S. Cl. CPC C07H 21/04; A61K 31/711; A61K 33/38; A61K 47/549; C12N 15/113; C12N 15/115; C12N 2310/14; C12N 2310/16; C12N 2310/3519; C12N 2310/531

(57) ABSTRACT

Described herein are RNA-DNA fusomers comprising a single-stranded polynucleotide comprising alternating RNA and DNA segments, wherein the polynucleotide self-assembles into a double-stranded DNA core with single-stranded RNA loops at either end of the fusomer, such as fusomers comprising three DNA segments surrounding two RNA segments. Also described herein are methods using said fusomers in laboratory and/or clinical settings.

### RELATED APPLICATION INFORMATION

This application claims the benefit of U.S. Provisional Application Ser. No. 63/663,827, filed Jun. 25, 2024, the disclosure of which is incorporated herein by reference in its entirety.

### STATEMENT OF GOVERNMENT SUPPORT

This invention was made with government support under Grant Numbers EB 032640 and GM 139587 awarded by The National Institutes of Health. The government has certain rights in the invention.

### STATEMENT REGARDING ELECTRONIC FILING OF A SEQUENCE LISTING

A Sequence Listing in XML format, entitled 9812-14_ST26.xml, 50,959 bytes in size, generated on Apr. 25, 2025, and filed herewith, is hereby incorporated by reference in its entirety for its disclosures.

### FIELD OF THE INVENTION

The present invention relates to RNA-DNA fusomers, i.e., self-assembling nucleic acid nanoparticles, and methods of use thereof.

### BACKGROUND

Rationally designed self-assembling nucleic acid nanoparticles (NA NPs) offer a unique class of functional therapeutics. They have structured designs that can be customized with gene silencing agents, targeting ligands, fluorophores, small molecule drugs, or any other cargos incorporated into the DNA and/or RNA scaffolds. NANPs have been extensively characterized for their potential use in a wide array of diseases, such as inflammatory diseases, cancers, viral infections, bacterial infections, and cardiovascular disorders (2). Recent studies confirmed that fibrous nucleic acid nanostructures are immunoquiescent as compared to planar or globular nucleic acid nanoparticles (3-5).

Hence, nucleic acid fibers have been functionalized for the delivery of siRNAs with a reduced immunological recognition (6). Another study has shown that nucleic acid nanoparticles, comprising short DNA or RNA strands, offer precise control over functionalization with therapeutic agents and modulation of immunological properties (7). In addition, the chemical composition of nucleic acid nanoparticles plays a major role in immunorecognition, where RNA based nanoparticles induce immunostimulation and the production of pro-inflammatory cytokines and interferons, while DNA counterparts are immunoquiescent in most cases (7-11).

Accordingly, there is a need in the art for DNA and/or RNA based nanoparticles which can be used in a diverse set of biomedical applications, such as gene silencing, protein downregulation, enzyme deactivation, bacterial growth inhibition, and in biosensing via solid-state nanopores.

### SUMMARY OF THE INVENTION

One aspect of the present invention is directed to an RNA-DNA fusomer comprising a single-stranded polynucleotide comprising alternating RNA and DNA segments, wherein the polynucleotide self-assembles into a double-stranded DNA core with single-stranded RNA loops at either end of the fusomer. In some embodiments, the polynucleotide comprises three DNA segments surrounding two RNA segments.

The fusomers may assemble into nucleic acid fibers through interaction of the RNA loops of different fusomers. Thus, another aspect of the invention is directed to a composition comprising two or more fusomers as described herein. In some embodiments, the first and/or second RNA loop of one fusomer hybridizes to the first and/or second RNA loop of a second fusomer and/or a third fusomer to assemble into a nucleic acid fiber.

Another aspect of the invention is directed to a method for modulating expression of a target nucleic acid molecule and/or protein in a cell, the method comprising contacting the cell with a fusomer of the present invention, wherein the cell expresses the nucleic acid and/or protein targeted by the fusomer.

Another aspect of the invention is directed to a method for deactivating a protein (e.g., an enzyme) in a cell, the method comprising contacting the cell with a fusomer of the present invention, wherein the fusomer binds to the protein and deactivates it.

Another aspect of the invention is directed to a method for inhibiting the growth of a microorganism, the method comprising contacting the microorganism with a fusomer of the present invention, thereby inhibiting the growth of the microorganism.

Another aspect of the invention is directed to a method for reducing blood coagulation in a subject, the method comprising administering to the subject a therapeutically effective amount of a fusomer of the present invention, thereby reducing blood coagulation.

Another aspect of the invention is directed to a method for modulating an immune response in a subject, the method comprising administering to the subject a therapeutically effective amount of a fusomer of the present invention, thereby modulating the immune response in the subject.

Another aspect of the invention is directed to a method for modulating an inflammatory response in a subject, the method comprising administering to the subject a therapeutically effective amount of a fusomer of the present invention, thereby modulating the inflammatory response in the subject.

Another aspect of the invention is directed to a method for detecting a target protein by a biosensing nanopore, the method comprising contacting the target protein with a fusomer of the present invention and determining the capture rate of the RNA-DNA fusomer by the biosensing nanopore, thereby detecting the target protein by the biosensing nanopore.

Another aspect of the invention is directed to a method for delivering a therapeutic agent to a subject, the method comprising contacting a fusomer of the present invention with the therapeutic agent, wherein the RNA-DNA fusomer binds to the therapeutic agent, and then administering the RNA-DNA fusomer that is bound to the therapeutic agent to the subject, thereby delivering the therapeutic agent to the subject.

These and other aspects of the present invention are set forth in more detail in the description of the invention below.

### BRIEF DESCRIPTION OF THE FIGURES

FIG. 1, panel A is a schematic outlining the benefits of the fusomer design as described herein. FIG. 1, panel B is a schematic outlining the fusomers design; the RNA monomer corresponds to SEQ ID NO: 7 and the RNA-DNA fusomer corresponds to SEQ ID NO: 1.

FIG. 1, panels C and D are schematics showing different architectural and functional parameters tested, the NF-κB decoy sequence corresponds to SEQ ID NOs: 34 and 35 for the top and bottom strand, respectively. FIG. 1, panel E is a schematic of the experimental outline to test fusomers. The dots indicate DNA nucleotides.

FIG. 2, panel A is a schematic outline of peripheral blood mononuclear cells (PBMCs) testing. FIG. 2, panel B is a schematic of nucleic acid nanoparticles tested including DNA cubes, RNA cubes, RNA fibers, RNA-fusomer fibers, and fusomer-fusomer fibers. FIG. 2, panel C is a series of graphs showing Type I and III IFN production levels of three healthy donors after treatment with DNA cubes, RNA cubes, RNA-RNA fibers, fusomers, and RNA-fusomers.

FIG. 3, panel A is a series of atomic force microscopy (AFM) topography images and corresponding histograms of the curvature values of RNA fibers and hybrid fusomers with varying stem lengths. FIG. 3, panel B is a heatmap showing the variation in lengths and compositions of fibers with tunable mechanical properties linked to immune stimulation in THP-1 dual reporter cells through nuclear factor kappa-light-chain-enhancer of activated B cells (NF-κB) and interferon regulatory factor (IRF) pathways, (n=3)

FIG. 4, Panel A is a schematic layout of a 3L 64 OrganoReady® Colon Caco-2 plate, displaying the channels in each chip, and demonstrating how the Caco-2 tubule is seeded against the collagen-I. FIG. 4, Panel B is a graph showing the transepithelial electrical resistance (TEER) measurements following treatment with RNA fibers and fusomers complexed with L2K. Control conditions include vehicle alone, and positive control that disrupts the barrier integrity. TEER values are presented as mean±SEM, (n=3). FIG. 4, Panel C is a series of representative microscopy images of OrganoReady® Colon Caco-2 cultures at 4× magnification, showing fluorescent uptake of RNA fibers and Fusomers after 24 hr. The intensity of the IRD800 signal was quantified and presented as mean±SD, (n=3) in the corresponding graph.

FIG. 5, Panel A is a schematic of fusomers functionalized with NF-κB decoys, their binding to NF-κB protein and nanopores analysis. FIG. 5, Panel B is a series of AFM topography images of fusomers and fusomers bound to NF-κB protein, and corresponding histograms of the curvature values. FIG. 5, Panel C is a series of graphs showing the characterization of NF-κB protein and its complex with NF-κB decoy fusomer: current trace (1) and scatter plot of fractional current blockade and dwell time (2) at 400 mV, current trace (3) and histogram of interevent time (4) at −400 mV for NF-κB protein. Current trace (5) and scatter plot of fractional current blockade and dwell time (6) at 400 mV (arrow shows the extra population of duplex-bound protein), current trace (7) and histogram of interevent time (8) at −400 mV for the complex of NF-κB protein with NF-κB decoy duplex monomer. The concentration of the protein in each sample was 71 nM. Current traces were recorded at a sampling rate of 4167 kHz and low pass filtered at 250 kHz.

FIG. 6, panel A is a schematic outlining the design of dual action fusomers, and their activity in the cell for NF-κB downregulation. FIG. 6, panel B is an AFM topography image of dual function fusomers, and corresponding histogram of the curvature value. FIG. 6, panel C is a series of graphs showing the cytokine production upon PBM Cs treatment with fusomers of dual function. FIG. 6, panel D is a schematic outline of reporter cell line testing and a series of heatmaps showing the immune recognition of the tested fusomers with NF-κB decoy and dual function fusomers assessed in reporter HEK-Blue hTLR3, HEK-Blue hTL R7, HEK-Blue hTLR9, HEK-Lucia RIG-I, and THP1-Dual cell lines (n=3).

FIG. 7, panel A is a schematic of the synthesis of DNA-templated silver nanoclusters (AgN Cs) attached fusomers. FIG. 7, panel B is an image showing the change of fluorescence of functionalized fusomers upon A gNCs synthesis. FIG. 7, panel C is an AFM topography image of A gN Cs fusomers, and corresponding histogram of the curvature value. FIG. 7, panel D is a series of EEM micrographs showing the fluorescence of fusomers AgNCs Vs C12 AgNCs. FIG. 7, panel E is a schematic showing the experimental protocol and a graph showing the K12 E. coli growth curves upon treatment with AgNCs fusomers (4-6 uM), carbenicillin, buffer, equivalent amounts of silver nitrate (silver control), and DNA control. FIG. 7, panel F is a schematic showing the experimental protocol and a graph showing the HEK 293-FT cell viability measured by MTS assay upon treatment with A gN Cs fusomers (4-6 uM), C 12 AgNCs (4-6 uM), carbenicillin, buffer, and DNA control. The results depicted as the average of three biological repeats with three technical repeats each.

FIG. 8, panel A is a schematic depiction of the anticoagulant effect of the Fusomers-NU 172 aptamer. FIG. 8, panel B is a series of graphs showing plasma coagulation assessment of NU fusomers using Prothrombin Time (PT), Activated Partial Thromboplastin Time (APTT), and Thrombin Time (TT) tests with human plasma. The results are depicted as the average of three biological repeats with three technical repeats each.

FIG. 9 is a series of histograms of the curvature values of A FM topography images of modified fusomers.

FIG. 10, panel A is a schematic outlining the dehydration of fibers and fusomers via lyophilization and vacuum drying, the different conditions of storage for a week, and rehydration and assessment of stability on Native-PAGE. FIG. 10, panel B is a series of Native-PAGE gels showing the stability of the dehydrated samples via lyophilization and vacuum drying at 55° C. as compared to solution control stored at the same temperature, and the positive control stored at 4° C.

FIG. 11, panel A is a series of heatmaps showing the immune recognition of the tested fibers and fusomers in the reporter HEK-Blue hTLR3, HEK-Blue hTLR7, HEK-Blue hTLR9, HEK-Lucia RIG-I, and THP1-Dual cells, controls were added in each experiment to activate the NF-κB or IRF pathway. FIG. 11, panel B is a series of graphs showing cell viability in six reporter cell lines.

FIG. 12, panel A is a graph showing the current trace of an RNA fiber (at 65 nM concentration) recorded at a sampling rate of 250 kHz and low pass filtered at 100 kHz. FIG. 12, panel B is a scatter plot showing the fractional current blockade and dwell time of an RNA fiber (at 65 nM concentration). FIG. 12, panel C is a graph showing the exponential fitting of histogram of interevent time of an RNA fiber (at 65 nM concentration). FIG. 12, panel D is a graph showing the current trace of a hybrid fiber with NF-κB decoy (at 71 nM concentration) recorded at a sampling rate of 4167 kHz and low pass filtered at 250 kHz. FIG. 12, panel E is a scatter plot showing the fractional current blockade and dwell time of a hybrid fiber with NF-κB decoy (at 71 nM concentration). FIG. 12, panel F is a graph showing the exponential fitting of histogram of interevent time of a hybrid fiber with NF-κB decoy (at 71 nM concentration). FIG. 12, panel G is a graph showing the current trace of a hybrid fiber with NF-κB decoy and DS RNA (at 71 nM concentration) recorded at a sampling rate of 4167 kHz and low pass filtered at 250 kHz. FIG. 12, panel H is a scatter plot showing the fractional current blockade and dwell time of a hybrid fiber with NF-κB decoy and DS RNA (at 71 nM concentration), arrows show the additional population resulting from the DS RNA branching. FIG. 12, panel I is a graph showing the exponential fitting of histogram of interevent time of a hybrid fiber with NF-κB decoy and DS RNA (at 71 nM concentration).

FIG. 13, panel A is a current trace for NF-κB decoy duplex monomer. FIG. 13, panel B is a current trace for NF-κB protein. FIG. 13, panel C is a current trace for a complex of NF-κB protein and a NF-κB decoy duplex monomer. For all panels, the current trace was acquired at 400 mV in 1M KCl, 10 mM HEPES, 2 mM M gCl₂ at pH 7.5. The pore used was 4.5-6.5 nm in size. The current traces were recorded at a sampling rate of 4167 kHz and low pass filtered at 250 kHz.

FIG. 14, panel A is a current trace for a hybrid fiber with NF-κB decoy. FIG. 14, panel B is a current trace for NF-κB protein. FIG. 14, panel C is a current trace for NF-κB protein and a hybrid fiber with NF-κB decoy complex prepared by mixing the protein and fiber in 2:1 ratio. For all panels, the current trace was recorded for 40 seconds at 200 mV in 1M KCl, 10 mM HEPES, 2 mM M gCl₂ at pH 7.5. The pore used was 8 nm in size. The current traces were recorded at a sampling rate of 4167 kHz and low pass filtered at 250 KHz.

FIG. 15, panel A is a current trace for a hybrid fiber with NF-κB decoy and dicer substrates (DS) RNA. FIG. 15, panel B is a current trace for 20% NF-κB protein (33.3 nM) and 80% hybrid fiber with NF-κB decoy and DS RNA. FIG. 15, panel C is a current trace for 30% NF-κB protein (33.3 nM) and 70% hybrid fiber with NF-κB decoy and DS RNA. For all panels, the current trace was recorded for 10 minutes at 150 mV in 0.4M K Cl, 10 mM HEPES, 2 mM M gCl₂ at pH 7.5. The pore used was 12 nm in size. The current traces were recorded at a sampling rate of 4167 kHz and low pass filtered at 250 KHz.

FIG. 16 is a series of microscopy images showing the qualitative analysis of green fluorescent protein (GFP) downregulation using GFP fusomers complexed with Lipofectamine 2000 (L2K) and RNA GFP dicer substrates (DS) also complexed with L2K at 10 nM and 50 nM concentrations. The analysis also includes a cells alone treatment. Each image panel includes two fields of visualization: brightfield and GFP, captured at a 10X objective.

### DETAILED DESCRIPTION

The present invention will now be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 C.F.R. § 1.831 and established usage.

### Definitions

The following terms are used in the description herein and the appended claims.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12,13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “enhance,” “enhancing,” “improve” and “improving” (and grammatical variations thereof) describe an elevation of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more such as compared to another measurable property or quantity (e.g., a control value).

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 30%, 35%, 40%, 45%, 50%, 55%, 60%, 65%, 70%, 75%, 80%, 85%, 90%, 95%, 97%, 98%, 99%, or 100% such as compared to another measurable property or quantity (e.g., a control value). In some embodiments, the reduction can result in no or essentially no (i.e., an insignificant amount, e.g., less than about 10% or even 5%) detectable activity or amount.

By the terms “treat,” “treating,” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing,” and “prevention” (and grammatical variations thereof) refer to prevention and/or delay of the onset of a disease, infection, disorder and/or a clinical symptom(s) in a subject and/or a reduction in the severity of the onset of the disease, infection, disorder and/or clinical symptom(s) relative to what would occur in the absence of the methods of the invention. The prevention can be complete, e.g., the total absence of the disease, infection, disorder and/or clinical symptom(s). The prevention can also be partial, such that the occurrence of the disease, infection, disorder and/or clinical symptom(s) in the subject and/or the severity of onset is less than what would occur in the absence of the present invention.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, infection, disorder and/or clinical symptoms in a subject and/or to reduce and/or delay the severity of the onset of a disease, infection, disorder and/or clinical symptoms in a subject relative to what would occur in the absence of the methods of the invention. Those skilled in the art will appreciate that the level of prevention need not be complete, as long as some benefit is provided to the subject.

The terms “complementary” or “complementarity,” as used herein, refer to the natural binding of polynucleotides under permissive salt and temperature conditions by base-pairing. For example, the sequence “A-G-T” (5′ to 3′) binds to the complementary sequence “T-C-A” (3′ to 5′). Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

“Complement” as used herein can mean 100% complementarity (i.e., fully complementary) with the comparator nucleotide sequence or it can mean less than 100% complementarity (e.g., “substantially complementary” or “partially complementary” such as about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like, complementarity).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence as compared to a reference polypeptide.

The term “administering” or “administration” of a composition of the present invention to a subject includes any route of introducing or delivering to a subject a compound to perform its intended function.

### Compositions

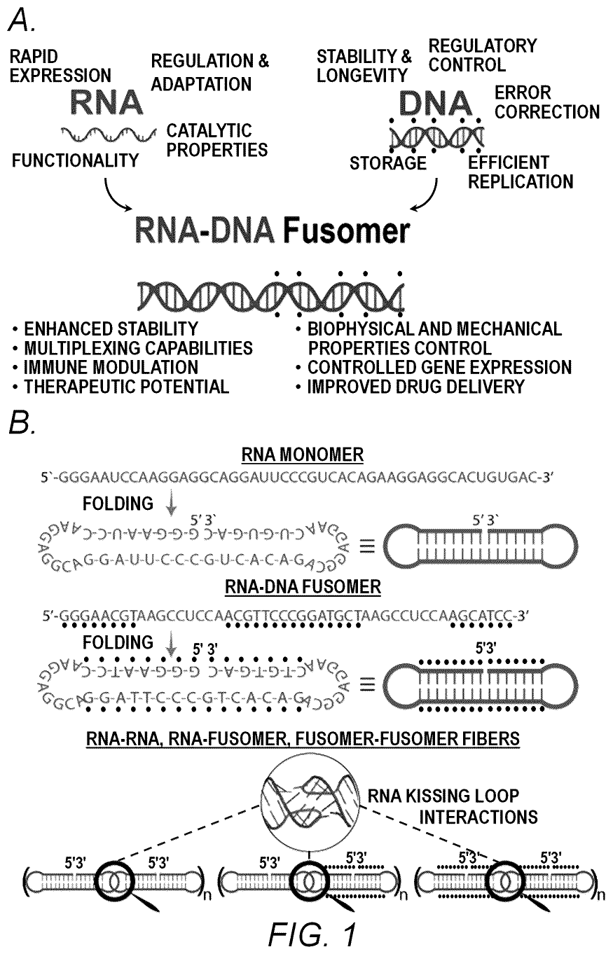

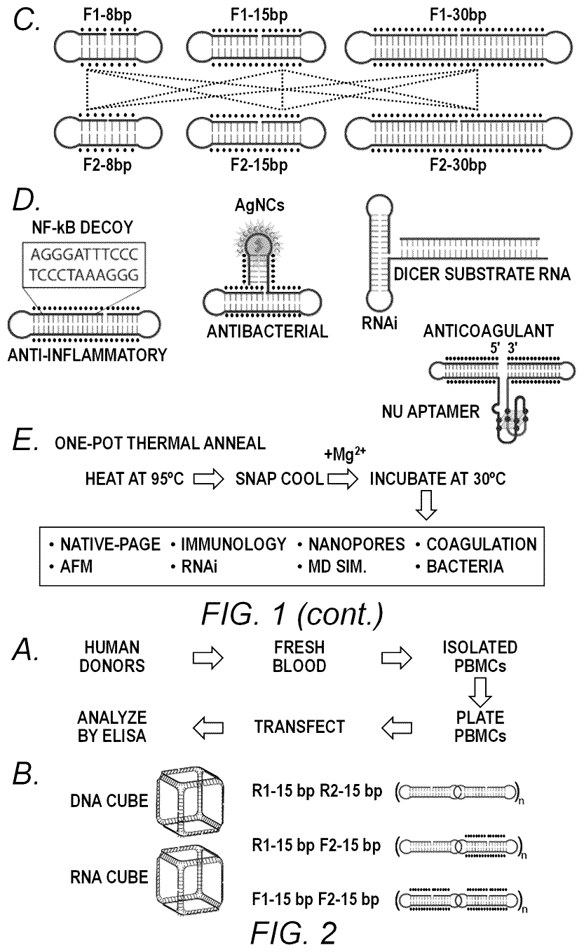

The present invention is based on the finding that one-dimensional hybrid RNA-DNA fibrous nucleic acid nanoparticles (e.g., fusomers) can act as a novel therapeutic and/or bind to known therapeutics to aid in their efficacy. Thus, one aspect of the invention relates to an RNA-DNA fusomer comprising a single-stranded polynucleotide comprising alternating RNA and DNA segments, wherein the polynucleotide self-assembles into a double-stranded DNA core with one single-stranded RNA loop at either end of the fusomer (e.g., the fusomer has a dumbbell shape). In some embodiments, each of the single-stranded RNA loops is independently between 8 and 20 nucleotides in length (e.g., between 8, 9, 10, 11, 12, 13, 14, 15, 16, 17, 18, 19, and 20 nucleotides in length). In some embodiments, the single-stranded RNA loops act as “kissing loops” and provide a biological function (e.g., have a specific binding capability for a different kissing loop on a second fusomer; have a specific binding capability for a therapeutic agent; have a specific binding capability for a target nucleic acid and/or protein). In some embodiments, the polynucleotide comprises three DNA segments surrounding two RNA segments. In some embodiments, the first and/or third DNA segments are complementary to the second DNA segment and base-pair thereto. In some embodiments, the fusomer self-assembles into a double-stranded DNA core with one single-stranded RNA loop at either end of the fusomer, and wherein the first and/or third DNA segment comprises one or more sequences that self-base pair to form one or more stem-loops (e.g., the fusomer has a clover shape as in FIG. 1, panel D). In some embodiments, the fusomer self-assembles into a double-stranded DNA core with one single-stranded RNA loop at either end of the fusomer, and wherein the first and/or third DNA segment has a tail that does not base pair to the second DNA segment (e.g., the fusomer has a “T” shape as in FIG. 1, panel D). In some embodiments, the tail of the first and/or third DNA segment base pairs to a separate DNA and/or RNA sequence (e.g., to form a DS RNA and/or DNA sequence as in FIG. 1, panel D).

In some embodiments, a fusomer comprises, consists essentially of, or consists of a nucleic acid sequence that is between 25 and 400 nucleotides in length (e.g., about 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, or 400 nucleotides in length). In some embodiments, a fusomer comprises, consists essentially of, or consists of a nucleic acid sequence having about 70% to about 99% sequence identity (e.g., about 70%, 75%, 80%, 85%, 90%, 91%, 92%, 93%, 94%, 95%, 96%, 97%, 98%, or 99% sequence identity) to any one of the nucleic acids of SEQ ID NOS: 1-6, 9, 14, 15, or 17. In some embodiments, a fusomer comprises, consists essentially of, or consists of the nucleic acid sequence of any one of SE Q ID NOS: 1-6, 9, 14, 15, or 17. In some embodiments, the first, second, and/or third DNA segment comprises, consists essentially of, or consists of a nucleic acid sequence having about 70% to about 99% sequence identity (e.g., about 70%, 75%, 80%, 85%, 90%, 91%, 92%, 93%, 94%, 95%, 96%, 97%, 98%, or 99% sequence identity) to any one of the nucleic acids of SE Q ID NOS: 12, 13, 16, 21-26, or 28-33, or having the sequence of GGGAATCC, CTGTGAC, GGGAACGT, or AGCATCC. In some embodiments, the first, second, and/or third DNA segment comprises, consists essentially of, or consists of the nucleic acid sequence of any one of SEQ ID NOS: 12, 13, 16, 21-26, 28-33, CGC, GGGA, GGGC, GCGAA, TCCCGCCC, GCGTTCGC, GGGAATCC, CTGTGAC, GGGAACGT, or AGCATCC. In some embodiments, the first and/or second RNA segment comprises, consists essentially of, or consists of a nucleic acid sequence having about 70% to about 99% sequence identity (e.g., about 70%, 75%, 80%, 85%, 90%, 91%, 92%, 93%, 94%, 95%, 96%, 97%, 98%, or 99% sequence identity) to any one of the nucleic acids having the sequence of AAGGAGGCA or AAGCCTCCA. In some embodiments, the first and/or second RNA segment comprises, consists essentially of, or consists of the nucleic acid sequence having the sequence of AAGGAGGCA or AAGCCTCCA.

In some embodiments, the first and/or third DNA segments are fully complementary to the second DNA segment (i.e., the first DNA segment is fully complementary to a first portion of the second DNA segment, and/or the third DNA segment is fully complementary to a second portion of the second DNA segment). In some embodiments, the first and/or third DNA segments are partially complementary to the second DNA segment (i.e., the first DNA segment is partially complementary to a first portion of the second DNA segment, and/or the third DNA segment is partially complementary to a second portion of the second DNA segment). In some embodiments, each of the DNA segments are independently between 4 and/or 60 nucleotides in length (e.g., between 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60 nucleotides in length or any range therein). In some embodiments, the first and/or second RNA segments are between 8 and 50 nucleotides in length (e.g., between 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50 nucleotides in length or any range therein). In some embodiments, the nucleic acid sequence of the first and second RNA segments is the same. In some embodiments, the third DNA segment is longer than the first DNA segment by about 4 to about 30 nucleotides (e.g., by about 4, 5, 6, 7, 8, 9, 10, 11, 12, 13, 14, 15, 16, 17, 18, 19, 20, 21, 22, 23, 24, 25, 26, 27, 28, 29, or 30 nucleotides or any range therein). In some embodiments, a portion of the first, second, and/or third DNA segment is self-complementary (e.g., fully self-complementary or partially self-complementary). In some embodiments, the self-complementary portion of the first, second, and/or third DNA segment self-hybridizes to form a stem-loop structure.

In some embodiments, one or more of the DNA segments further comprises a sequence of a therapeutic nucleic acid (TNA). Example TNA sequences include, but are not limited to, small interfering RNA (siRNA), RNA (e.g., mRNA) and/or DNA aptamers, antisense oligonucleotides, peptide nucleic acids, DNA zymes, ribozymes, RNA and/or DNA decoys, xeno nucleic acids (XNA), dicer substrate RNA (DS RNA), and the like. Example TNA sequences embedded in the one or more DNA segments include, but are not limited to, one or more of the nucleic acid sequences of SEQ ID NOs: 12, 13, 16, and/or 27.

In some embodiments, the aptamer is an anticoagulant (e.g., antithrombin) aptamer (e.g., a NU 172 aptamer comprising the nucleic acid sequence of SEQ ID NO: 27).

In some embodiments, the RNA and/or DNA decoy is a decoy for an inflammatory cytokine (e.g., NF-κB, tumor necrosis factor alpha (TNFα), interleukin-1a (IL-1a), and/or interleukin-6 (IL-6)) (e.g., the fusomer acts as an anti-inflammatory agent). In some embodiments, the RNA and/or DNA decoy is a NF-κB decoy having the nucleic acid sequence of one or more of SEQ ID NOs: 11-13 and/or the reverse complement thereof. In some embodiments, the RNA and/or DNA decoy is a green fluorescent protein (GFP) decoy having the nucleic acid sequence of SEQ ID NOs: 19 or 20 and/or the reverse complement thereof.

In some embodiments, the TNA sequence is between about 4 to about 50 nucleic acids in length (e.g., about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleic acids in length or any range therein).

In some embodiments, the fusomer is capable of binding to a therapeutic agent. In some embodiments, one or more structural elements (e.g., one or more stem-loops) in the fusomer creates a binding interface for the therapeutic agent. In some embodiments, the fusomer comprises a nucleic acid sequence that is capable of binding to a therapeutic peptide. In some embodiments, the therapeutic agent is a TNA sequence and the fusomer comprises a nucleic acid sequence that is complementary to, and thus hybridizes, the TNA sequence.

In some embodiments, the stem-loop structure is capable of binding to a therapeutic agent. In some embodiments, the therapeutic is an antimicrobial agent. In some embodiments, the antimicrobial agent is a silver nanocluster (AgNC) (i.e., a silver nanoparticle (AgNP)). In some embodiments, the fusomer comprises a cytosine-rich hairpin stem-loop (e.g., a C12 hairpin) that binds to the A gNC. In some embodiments, the antimicrobial agent is an antibiotic (e.g., penicillin, streptomycin, ampicillin, cephalosporin, tetracycline, doxycycline, amoxicillin, vancomycin, and the like).

As used herein, the term “non-toxic” is intended to mean a substance or composition that, when administered to a population of cells does not increase cell death in said population of cells by more than 20% when compared to a control population of cells that have not received administration of the substance or composition; additionally, as used herein, the term “non-toxic” is intended to mean a substance that is susceptible to approval by the United States Federal Drug Administration for administration to mammals, preferably humans. In some embodiments, the fusomer is non-toxic to mammalian cells. In some embodiments, the fusomer is non-toxic to avian cells. In some embodiments, the fusomer is non-toxic to fish cells. In some embodiments, the fusomer is non-toxic to reptile cells. In some embodiments, the fusomer is non-toxic to amphibian cells. In some embodiments, the fusomer is non-toxic to insect cells. In some embodiments, the fusomer is non-toxic to bacterial cells. In some embodiments, the fusomer is non-toxic to yeast cells.

In some embodiments, one or more fusomers as described herein may be in a composition (e.g., a pharmaceutical composition) for use in the methods as described herein.

Another aspect of the invention relates to composition comprising two or more RNA-DNA fusomers as described herein. In some embodiments, one RNA segment of one RNA-DNA fusomer (e.g., a first RNA kissing loop) hybridizes to one RNA segment of a second RNA-DNA fusomer (e.g., a second RNA kissing loop) and/or a third RNA-DNA fusomer (e.g., a third RNA kissing loop). In some embodiments, the hybridized RNA-DNA fusomers assemble into one or more nucleic acid fibers. In some embodiments, the nucleic acid fiber comprises two fusomers (e.g., two dumbbell shaped fusomers) wherein a first RNA loop on a first fusomer is hybridized to a second RNA loop on a second fusomer (as in FIG. 1, panel b).

Another aspect of the invention relates to composition comprising an RNA-DNA fusomer as described herein and an RNA fiber (e.g., a partially self-complementary RNA sequence that is capable of self-assembly into multiple stem-loop structures that mimics the structure of one or more fusomers as described herein). In some embodiments, an RNA fiber comprises, consists essentially of, or consists of a nucleic acid sequence having about 70% to about 99% sequence identity (e.g., about 70%, 75%, 80%, 85%, 90%, 91%, 92%, 93%, 94%, 95%, 96%, 97%, 98%, or 99% sequence identity) to any one of the nucleic acids of SEQ ID NOs: 7, 8, 10, or 18. In some embodiments, a fusomer comprises, consists essentially of, or consists of the nucleic acid sequence of any one of SEQ ID NOS: 7, 8, 10, or 18.

### Methods of Use

Also provided herein are methods of using RNA-DNA fusomers and compositions of RNA-DNA fusomers as described herein. Thus, another aspect of the invention relates to a method for modulating expression of a target nucleic acid molecule and/or protein in a cell, the method comprising contacting the cell with an RNA-DNA fusomer as described herein or a composition as described herein wherein the cell expresses the nucleic acid and/or protein targeted by the RNA-DNA fusomer. In some embodiments, the fusomer comprises a DS RNA and/or DNA sequence; an RNA i sequence; and/or an antisense oligonucleotide sequence that hybridizes to the target nucleic acid molecule and prevents translation and/or degrades (e.g., by activating the Dicer protein) the target nucleic acid molecule, thereby modulating the expression of the target nucleic acid molecule and/or protein. In some embodiments, the expression of the nucleic acid and/or protein is modulated (e.g., decreased) by about 5% to about 100% (e.g., about 5%, 10%, 15%, 20%, 25%, 30%, 35%, 40%, 45%, 50%, 55%, 60%, 65%, 70%, 75%, 80%, 85%, 90%, 95%, or 100%) compared to the expression of the nucleic acid and/or protein in a cell that is not contacted by the fusomer or composition of the present invention. In some embodiments, the expression of the nucleic acid and/or protein is modulated (e.g., increased) by about 5% to about 200% or more (e.g., about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, or more) compared to the expression of the nucleic acid and/or protein in a cell that is not contacted by the fusomer or composition of the present invention.

Another aspect of the invention relates to a method for deactivating a protein (e.g., an enzyme) in a cell, the method comprising contacting the cell with an RNA-DNA fusomer as described herein or a composition as described herein, wherein the RNA-DNA fusomer binds to the protein and deactivates it (e.g., reduces its biological activity). In some embodiments, the fusomer comprises a binding sequence for the target protein (e.g., a decoy) and binds to the target protein, thereby deactivating the protein. In some embodiments, the fusomer comprises an aptamer sequence that binds to the target protein and reduces its biological activity, thereby deactivating the protein. In some embodiments, the activity of the protein in the cell is reduced by about 5% to about 100% (e.g., about 5%, 10%, 15%, 20%, 25%, 30%, 35%, 40%, 45%, 50%, 55%, 60%, 65%, 70%, 75%, 80%, 85%, 90%, 95%, or 100%) compared to the activity of a protein in a cell that is not contacted by the fusomer or composition of the present invention.

Another aspect of the invention relates to a method for reducing or inhibiting the growth of a microorganism, the method comprising contacting the microorganism with an RNA-DNA fusomer as described herein or a composition as described herein, thereby reducing or inhibiting the growth of the microorganism. In some embodiments, the fusomer comprises a nucleic acid sequence (e.g., a C12 hairpin) that binds to an anti-microbial agent (e.g., an A gNC) as described herein, thereby reducing or inhibiting the growth of the microorganism. In some embodiments, the growth of the microorganism is reduced or inhibited by about 5% to about 100% (e.g., about 5%, 10%, 15%, 20%, 25%, 30%, 35%, 40%, 45%, 50%, 55%, 60%, 65%, 70%, 75%, 80%, 85%, 90%, 95%, or 100%) compared to a control microorganism that is not contacted by the fusomer or composition of the present invention.

Another aspect of the invention relates to a method for reducing blood coagulation in a subject, the method comprising administering to the subject a therapeutically effective amount of an RNA-DNA fusomer as described herein or a composition as described herein, thereby reducing blood coagulation. In some embodiments, the fusomer comprises an aptamer (e.g., NU 172) that binds to a blood coagulant (e.g., thrombin) and/or a procoagulant and reduces the biological activity of (e.g., inactivates) said blood coagulant and/or a procoagulant, thereby reducing blood coagulation. In some embodiments, the blood coagulation is reduced or inhibited by about 5% to about 100% (e.g., about 5%, 10%, 15%, 20%, 25%, 30%, 35%, 40%, 45%, 50%, 55%, 60%, 65%, 70%, 75%, 80%, 85%, 90%, 95%, or 100%) in the subject compared to a control subject that is not administered the fusomer or composition of the present invention.

Another aspect of the invention relates to a method for modulating an immune response in a subject, the method comprising administering to the subject a therapeutically effective amount of an RNA-DNA fusomer as described herein or a composition as described herein, thereby modulating the immune response in the subject. In some embodiments, the fusomer comprises an RNA and/or DNA aptamer or an RNA and/or DNA decoy that binds to an immune response protein (e.g., NF-κB) and reduces the biological activity of (e.g., inactivates) said immune response protein, thereby modulating the immune response. In some embodiments, the fusomer comprises an siRNA and/or DS RNA sequence that binds to an mRNA that encodes an immune response protein (e.g., NF-κB) and reduces or increases the expression of said immune response protein, thereby modulating the immune response. In some embodiments, the immune response is modulated (e.g., decreased) by about 5% to about 100% (e.g., about 5%, 10%, 15%, 20%, 25%, 30%, 35%, 40%, 45%, 50%, 55%, 60%, 65%, 70%, 75%, 80%, 85%, 90%, 95%, or 100%) in the subject compared to a control subject that is not administered the fusomer or composition of the present invention. In some embodiments, the immune response is modulated (e.g., increased) by about 5% to about 200% or more (e.g., about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, or more) in the subject compared to a control subject that is not administered the fusomer or composition of the present invention.

Another aspect of the invention relates to a method for modulating an inflammatory response in a subject, the method comprising administering to the subject a therapeutically effective amount of an RNA-DNA fusomer as described herein or a composition as described herein, thereby modulating the inflammatory response in the subject. In some embodiments, the fusomer comprises an RNA and/or DNA aptamer or an RNA and/or DNA decoy that binds to an inflammatory response protein (e.g., NF-κB) and reduces the biological activity of (e.g., inactivates) said inflammatory response protein, thereby modulating the inflammatory response. In some embodiments, the fusomer comprises an siRNA and/or DS RNA sequence that binds to an mRNA that encodes an inflammatory response protein (e.g., NF-κB) and reduces or increases the expression of said inflammatory response protein, thereby modulating the inflammatory response. In some embodiments, the inflammatory response is modulated (e.g., decreased) by about 5% to about 100% (e.g., about 5%, 10%, 15%, 20%, 25%, 30%, 35%, 40%, 45%, 50%, 55%, 60%, 65%, 70%, 75%, 80%, 85%, 90%, 95%, or 100%) in the subject compared to a control subject that is not administered the fusomer or composition of the present invention. In some embodiments, the inflammatory response is modulated (e.g., increased) by about 5% to about 200% or more (e.g., about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, or more) in the subject compared to a control subject that is not administered the fusomer or composition of the present invention.

Another aspect of the invention relates to a method for detecting a target protein by a biosensing nanopore, the method comprising contacting the target protein with an RNA-DNA fusomer as described herein or a composition as described herein and determining the capture rate of the RNA-DNA fusomer by the biosensing nanopore, thereby detecting the target protein by the biosensing nanopore. In some embodiments, the method comprises providing two fluid compartments separated by an electrically resistant membrane bilayer including a biosensing nanopore, introducing a sample comprising the fusomer and/or target protein, to a first fluid compartment, and applying an electric field across the membrane. In some embodiments, the electrical current (e.g., a current modulation signature) of the sample is monitored across the membrane, from which a movement of the fusomer and/or target protein through the nanopore (e.g., from the first fluid compartment into the second fluid compartment) can be determined as a change in the current measured through the nanopore (e.g., as function of the sample current modulation signature). In some embodiments, the RNA-DNA fusomer comprises a sequence that binds to the target protein.

Another aspect of the invention relates to a method for delivering a therapeutic agent to a subject, the method comprising contacting an RNA-DNA fusomer as described herein or a composition as described herein with the therapeutic agent, wherein the RNA-DNA fusomer binds to the therapeutic agent, and then administering the RNA-DNA fusomer that is bound to the therapeutic agent to the subject thereby delivering the therapeutic agent to the subject. In some embodiments, the therapeutic agent is a TNA sequence. In some embodiments, the therapeutic is an antimicrobial agent (e.g., a silver nanocluster and/or an antibiotic).

In some embodiments, the hybrid RNA-DNA fibrous nucleic acid nanoparticles (e.g., fusomers) have a reduced immunostimulatory response when administered to a subject as compared to an RNA-only nucleic acid nanoparticle having a similar, or the same, sequence as the fusomer. In some embodiments, the reduced immunostimulatory response of the fusomer is reduced by about 5% to about 95% when compared to the RNA-only nucleic acid nanoparticle (e.g., about 5%, 10%, 15%, 20%, 25%, 30%, 35%, 40%, 45%, 50%, 55%, 60%, 65%, 70%, 75%, 80%, 85%, 90%, or 95% reduced immunostimulatory response). In some embodiments, the hybrid RNA-DNA fibrous nucleic acid nanoparticles (e.g., fusomers) have a reduced immunostimulatory response when administered to a subject as compared to an DNA-only nucleic acid nanoparticle having a similar, or the same, sequence as the fusomer. In some embodiments, the reduced immunostimulatory response of the fusomer is reduced by about 5% to about 95% when compared to the DNA-only nucleic acid nanoparticle (e.g., about 5%, 10%, 15%, 20%, 25%, 30%, 35%, 40%, 45%, 50%, 55%, 60%, 65%, 70%, 75%, 80%, 85%, 90%, or 95% reduced immunostimulatory response).

### Subjects, Pharmaceutical Formulations, and Modes of Administration

The methods of the present invention find use in both veterinary and medical applications. Suitable subjects include avians, reptiles, amphibians, fish, and mammals. The term “mammal” as used herein includes, but is not limited to, humans, primates, non-human primates (e.g., monkeys and baboons), cattle, sheep, goats, pigs, horses, cats, dogs, rabbits, rodents (e.g., rats, mice, hamsters, and the like), etc. Human subjects include neonates, infants, juveniles, and adults. Optionally, the subject is “in need of” the methods of the present invention, e.g., because the subject has or is believed at risk for a disease, infection, and/or disorder where administering a fusomer and/or composition as described herein would produce some beneficial therapeutic effect. As a further option, the subject can be a laboratory animal and/or an animal model of disease. Preferably, the subject is a human.

In particular embodiments, the present invention provides one or more pharmaceutical compositions comprising one or more fusomers as described herein in a pharmaceutically acceptable carrier and, optionally, other medicinal agents, pharmaceutical agents, stabilizing agents, buffers, carriers, adjuvants, diluents, etc. For injection, the carrier will typically be a liquid. For other methods of administration, the carrier may be either solid or liquid. In some embodiments, the pharmaceutically acceptable carrier is phosphate buffered saline (PBS) or water. As used herein, the term “stabilizing agent” is intended to mean an addition to a pharmaceutical composition that increases the duration of the shelf life of the composition, increases the range of acceptable storage temperatures of the composition, increases the retention of the composition on ocular surface and/or in the targeted organs, and/or reduces degradation of the composition that may be caused by repeated freeze/thaw cycles. Example stabilizing agents include, but are not limited to a polysaccharide (e.g., carboxymethylcellulose (CMC), chitosan, hyaluronic acid, pectin, starch, oxidized dextran, alginate, etc.) and/or a peptide (e.g., albumin, collagen, etc.).

By “pharmaceutically acceptable” it is meant a material that is not toxic or otherwise undesirable, i.e., the material may be administered to a subject without causing any undesirable biological effects.

The one or more fusomers and/or compositions as described herein may be administered to a subject by any suitable method including, but not limited to, intranasal administration (e.g., sprays or gels); oral administration (e.g., tablets, capsules, lozenges, sprays, or gels), e.g., sublingual sprays or gels; anal administration (e.g., gels or suppositories); ocular administration (e.g., eye drops or gels); intramuscular administration (e.g., intramuscular injection); subcutaneous administration (e.g., subcutaneous injection); subdermal administration (e.g., subdermal injection); intravenous administration (e.g., intravenous injection); and/or vaginal administration (e.g., gels or suppositories).

The amount of the disclosed compositions administered to a subject will vary from subject to subject, depending on the nature of the disclosed compositions and/or formulations, the species, gender, age, weight and general condition of the subject, the mode of administration, and the like. Effective dosages and schedules for administering the compositions may be determined empirically, and making such determinations is within the skill in the art. The dosage ranges for the administration of the disclosed compositions are those large enough to produce the desired effect (e.g., to treat a pathogen infection, to modulate an immune response, to reduce inflammation, and the like). The dosage should not be so large as to outweigh benefits by causing extensive or severe adverse side effects, such as unwanted cross-reactions, anaphylactic reactions, and the like, although some adverse side effects may be expected. The dosage can be adjusted by the individual clinician in the event of any counterindications. In some embodiments, the disclosed compositions and/or formulations are administered to the subject at a concentration of one or more fusomers ranging from 0.5 μM to 900 μM or more (e.g., about 0.5, 1, 2, 3, 4, 5, 10, 15, 20, 25, 30, 40, 50, 60, 70, 80, 90, 100, 200, 300, 400, 500, 600, 700, 800, or 900 μM per dose). In some embodiments, the disclosed compositions and/or formulations are administered to the subject at a concentration of one or more fusomers ranging from 1 mM to 5 mM or more (e.g., about 1, 2, 3, 4, or 5 mM per dose). Dosages above or below the range cited above may be administered to the individual subject if desired. The compositions can be administered in any herein disclosed pharmaceutical composition comprising a pharmaceutically acceptable carrier.

In some embodiments, the one or more fusomers are effective within about 5 minutes to about 60 minutes after administering (e.g., about 5, 10, 15, 20, 25, 30, 35, 40, 45, 50, 55, or 60 minutes). In some embodiments, the one or more fusomers are effective for about 3 hours to about 24 hours after administering (e.g., about 3, 3.5, 4, 4.5, 5, 5.5, 6, 7, 8, 9, 10, 11, 12, 13,14, 15, 16, 17, 18, 19, 20, 21, 22, 23, or 24 hours). In some embodiments, the one or more fusomers are effective for about 1 day to about 7 days or more after administering (e.g., about 1, 2, 3, 4, 5, 6, 7 days or more). In some embodiments, the one or more fusomers are administered one time per day, two times per day, three times per day, four times per day, five times per day, or more. In some embodiments, the one or more fusomers are administered one time per week, two times per week, three times per week, four times per week, five times per week, or more.

In some embodiments, a composition of the present invention is administered to a subject in an amount of about 1 μL to about 5 ml (e.g., about 1 μL, 2 μL, 3 μL, 4 μL, 5 μL, 10 μL, 15 μL, 20 μL, 25 μL, 30 μL, 35 μL, 40 μL, 45 μL, 50 μL, 100 μL, 150 μL, 200 μL, 250 μL, 300 μL, 350 μL, 400 μL, 450 μL, 500 μL, 600 μL, 700 μL, 800 μL, 900 μL, 1 mL, 2 mL, 3 mL, 4 mL, to about 5 mL).

Having described the present invention, the same will be explained in greater detail in the following examples, which are included herein for illustration purposes only, and which are not intended to be limiting to the invention.

### EXAMPLES

### Example 1

In this study, a new generation of an immunoquiescent hybrid, one-dimensional (1D) RNA-DNA fusomer is introduced where one sequence blends RNA and DNA oligonucleotides (FIG. 1, panel A and FIG. 1, panel B). The unique chemically synthesized fusomer brings together the functions of two crucial biopolymers in a single structure, allowing for the selective orchestration of warranted responses in human cells. This platform fuses the manipulable characteristics of RNA and DNA (12), introducing fusomers with blended advantageous characteristics of both polymers, such as enhanced stability, multiplexing capabilities, controlled gene expression, and controlled physicochemical properties (FIG. 1, panel A). Fusomers exploit the RNA's ability to form non-canonical interactions and forming biologically relevant motifs, along with the stability of DNA base pairing. Our approach advances nucleic acid nanotechnologies by introducing a unique concept: chemically synthesized RNA-DNA sequences that fold using DNA segments to expose 9-nt RNA interacting motifs (RNA kissing loops) on the sides of double-stranded DNAs (dsDNA s). The dsDNA stem carries an embedded functional therapeutic nucleic acid (TNA), while the HIV-I RNA kissing loops are maintained for long range interactions needed for the assembly of the structure (13).

This study assessed the physicochemical properties of the newly developed fusomers in which the stability, mechanical properties, and immunological properties of the system were investigated. 3D organ-on-a-chip models were used to assess the cellular uptake of fusomers using different carriers. Added to this, Molecular Dynamics simulations were also employed to further characterize the fusomers. Altogether, each of these components provide a comprehensive profile for the fusomers that can be exploited for different applications. The research also assessed fusomers for their ability to achieve significant synergistic effects by delivery of multiple therapeutic functionalities embedded and incorporated into their structure. This novel platform provides a higher localized concentration of the therapeutic moiety, easier implementation of various functionalities, enhanced tunability of physicochemical properties, and cost-effective production.

The work included in this study provided new insights on using the functionalized fusomers in various applications in mammalian and bacterial cells, such as gene silencing, downregulation of proteins, anticoagulation, and bacterial growth inhibition. In addition, one of the explored benefits of fusomers is their use in biosensing proteins. Our study provides insights on the versatility of the fusomers platform by varying the architectural and functional parameters. FIG. 1 provides an overview on the workflow for this study, outlining the design of fusomers (FIG. 1, panel B), different architectural and functional parameters tested (FIG. 1, panel C and FIG. 1, panel D), and the experimental roadmap (FIG. 1, panel E).

### Materials and Methods

All oligonucleotides were purchased from Integrated DNA Technologies (IDT), Inc. DNA Clean and Concentrator Kit was from Zymo Research. MyTaq Mix, 2X was obtained from Bioline. RQ1 RNase-Free DNase (1 μg/μL) was obtained from Promega. Dulbecco's Modified Eagle Medium (DMEM), RPMI 1640, 0.25% trypsin-EDTA, and phosphate buffered saline (PBS) were from Thermo Fisher Scientific, Inc. Fetal Bovine Serum (FBS) was obtained from R & D Systems, Inc. Penicillin-streptomycin solution, and HyPure cell culture grade water were from Cytiva/GE Heathcare Life Sciences, Inc. Lipofectamine™ 2000 Reagent (L2K) was obtained from Invitrogen, Inc. Sodium borohydride (NaBH4) was from TCI America, Inc. K 12 E. coli, and Luria broth (LB) were purchased from Sigma-Aldrich, Inc. CellTiter 96® A Queous One Solution Cell Proliferation Assay (MTS) was obtained from Promega, Inc. Temperature Gradient Gel Electrophoresis (TGGE) thermal block (Biometra GmbH).

### Design of RNA-DNA Fusomers

RNA-DNA fusomers are a hybrid fiber system designed to elicit various therapeutic effects simultaneously. The RNA-DNA monomers fold into a dumbbell shape comprised of the 9-nt long RNA interacting motifs (kissing loops) on the sides of dsDNA stem, that can vary in length and carry specific functionalities.

Three different stem lengths were designed: fusomers with 8, 15, and 30-nt dsDNA stems. In addition, fusomers with embedded NF-κB decoy sequences were designed for protein binding experiments via nanopore analysis. RNA fibers functionalized with DS RNAs against NF-κB or GFP were designed for protein silencing activity. Fusomer with C12 hairpin functionality for binding silver nanoclusters (A gNCs) was designed for antibacterial activity. Finally, fusomer with NU 172 aptamer was designed as an anticoagulant. Generally, the fiber structure is composed of 2 monomers (a,b system) that would specifically interact together forming the kissing loops. This provided the ability to design multifunctional fusomers, where one monomer will hold functionality different than the other monomer.

### Fusomers Assembly and Characterization (Native-PAGE and A FM)

RNA monomers were obtained by amplifying DNA templates via PCR using MyTad Mix. The PCR product was purified using the DNA Clean and Concentrator kit. Then, in vitro run-off transcription using T7 RNA Polymerase in 80 mM HEPES-KOH (pH 7.5), 50 mM DTT, 25 mM M gCl₂, 2.5 mM spermidine, and 5 mM of each rNTP at 37° C. over 3.5 h took place. RQ1 RNase-Free DNase was added to stop transcription, and sample was further incubated at 37° C. for 30 min. The RNA strands were purified via denaturing polyacrylamide gel electrophoresis (PAGE, 8%) (8 mM urea), the PAGE was run at 13 W for 2 h, in 89 mM tris-borate, 2 mM EDTA (TBE, pH 8.2). UV shadowing was used to visualize and excise the RNA bands, which were then eluted in TBE (pH 8.2), 300 mM NaCl at 4° C. overnight. RNA strands were precipitated by mixing the elution with 2.5 volumes of 100% EtOH and incubated for 4 h at −20° C. The mixtures were centrifuged at 10.0 G at 4° C. for 30 min, then 90% EtOH was used to wash the pellet by centrifuging at 10.0 G for 10 min at 4° C., the wash step was repeated 2 times. A CentriV ap micro-IR vacuum concentrator (Labconco) was used to vacuum dry the pelleted samples at 55°° C. with IR. The dried RNA samples were dissolved in HyPure cell culture grade water, and their concentrations were measured by NanoDrop 2000 (ThermoFisher) at 260 nm. The RNA strands were stored until use at −20° C.

For assembly of fibers and fusomers, oligonucleotide strands were combined in equimolar ratios with HyPure cell culture-grade water and assembled in a one-pot thermal anneal, by heating to 95° C. for 2 min, snap-cooling on ice for 2 min, then adding the assembly buffer (89 mM tris-borate (pH 8.2), 2 mM M gCl₂, 50 mM K Cl), and incubating for 20 min at room temperature. Fusomers were stored at 4° C. until use. For assembly of fusomer with NU 172 aptamer, the previously established protocol was followed.

To confirm successful assembly of fusomers, 8% native-PAGE (37.5:1 acrylamide: bis-acrylamide) was used for visualization. A Mini-PROTEAN Tetra Cell system (Bio-Rad) was used to prepare the gel, then the gel was pre-run at 150 V for 10 min, with running buffer (89 mM TB (pH 8.2), and 2 mM MgCl₂). To load samples, 2 μL of loading buffer (Assembly buffer, 30% glycerol, bromophenol blue, xylene cyanol), was mixed with 2 μL of each sample, and 4 μL were loaded per well. The gel was run for 30 min at 300 V at 4° C. cold room. Ethidium bromide (EtBr, 0.5 μg mL-1) was used to stain the gel for 5 min, then the gel was washed twice with double-deionized water (ddiH 20). A ChemiDoc MP (Bio-Rad) was used to image the gel.

The structure of fusomers were imaged using Atomic Force Microscopy (AFM). AFM topography imaging was performed on a freshly cleaved mica surface modified with 1-(3-aminopropyl) silatrane (14). Tapping mode was used for the imaging utilizing the M ultiM ode AFM Nanoscope IV system (Bruker Instruments) (7, 15).

For fusomers functionalized with C12 Hairpin (hp), the monomers were combined in equimolar ratio with HyPure cell culture-grade water and assembled in a one-pot thermal anneal, by heating to 95° C. for 2 min, snap-cooling on ice for 2 min, then adding the assembly buffer (500 mM NH₄OAc (pH 6.9), 10 mM M gCl₂), and incubating for 20 min at room temperature. Fusomers assembly was confirmed on 8% native-PAGE (37.5:1 acrylamide: bis-acrylamide) as described previously. Fusomers were stored at 4° C. until needed for AgNCs synthesis. For A gN Cs synthesis with the fusomer-C12 hp, 75 μM of fusomer-C12 hp, HyPure cell culture-grade water, 1 mM AgNO₃, and 20 mM NH₄OAc (pH 6.9) were combined, mixed and centrifuged, then incubated at 95° C. for 2 min. Following that, the solutions were incubated on ice for 20 min. 10 mM NaBH₄ solution was prepared fresh using chilled water and kept on ice. Equimolar concentration of NaBH₄ to Ag⁺ in the Fusomer AgNCs samples was used to treat the samples. C12 AgNCs were prepared by replacing fusomer with C12 hairpin DNA, while silver control solution was prepared by replacing the fusomer volume with HyPure cell culture-grade water. All samples were stored away from light at 4° C. for ˜16 hours (16, 17). Finally, the synthesis of AgNCs was confirmed by visualizing the samples via UV transilluminator.

### AFM Sample Preparation

Freshly cleaved mica was immersed in 167 μM aqueous solution of 1-(3-aminopropyl)-silatrane (APS) for 30 min (14, 18). The APS modified mica pieces were rinsed in deionized water and dried under a stream of ultra-high purity argon (AR UHP) and further dried under vacuum for at least 12 hours. RNA and RNA/DNA hybrid samples were diluted at 4° C. with assembly buffer and immediately deposited onto APS-modified mica for 2 min from the buffered solution (assembly buffer) using the “droplet method”, rinsed briefly with deionized water, and dried with a gentle flow of argon (19). Various concentrations were deposited and imaged. This was necessary to achieve desired coverage and avoid as much as possible the overlapping of apparent fiber nanostructures for further image analysis of curvature, end to end distance, REE, and contour length, LC.

### AFM Imaging and Image Analysis

Imaging was performed with MultiMode AFM Nanoscope IV system (Bruker Instruments, Santa Barbara, CA, USA) in Tapping Mode at ambient conditions. The images were recorded with a scanning rate of 1.5 Hz using an RTESPA-300 probe (Bruker Nano Inc., CA, USA) with a resonance frequency of ˜320 KHz and a spring constant of ˜40 N/m. Images were processed using the FemtoScan Online software package (Advanced Technologies Center, Moscow, Russia) (19, 20).

The analysis of curvature for the resultant nanostructures was done using a “Kappa” plugin in Fiji (21). First, the length scale of each image was calibrated from pixel to nm. Then, each fiber nanostructure was manually traced with an open B-spline curve. “Kappa” automatically fit the curve to topology of individual fibers and returned length of each fiber and the values of curvature at every point on the curve. Custom home-written Python code was used to calculate the end-to-end distance by identifying the starting point and ending point of a fiber. Statistical histograms of the data points were plotted with MagicPlot Pro software and further fitted by Gaussian functions.

### Stability of Fusomers

### Fetal Bovine Serum Stability Assay

Folded RNA fiber monomer and fusomer monomer (1 μM in AB pH 8.0) were mixed and incubated with 20% (v/v) fetal bovine serum (FBS) solution at 37° C. At each timepoint, 5 μL were aliquoted and mixed with 5 μL native-PAGE loading buffer (Assembly buffer, 30% glycerol, bromophenol blue, xylene cyanol), and immediately placed on dry ice to stop the reaction. The samples were loaded in reverse time order and analyzed by 8% native page at 4° C. Ethidium bromide (EtBr, 0.5 μg mL-1) was used to stain the gel for 5 min, then the gel was washed twice with double-deionized water (ddiH 20). A ChemiDoc MP (Bio-Rad) system was used to image the gel. Image LabTM Software was use for the analysis and comparison of intensity of bands of treated samples with corresponding untreated monomers to determine relative degradation.

### Dehydration and Stability

The stability of different fibers was assessed upon dehydration and rehydration following the protocol (5) with minor modifications. Two dehydration methods were tested: lyophilization and vacuum concentration. RNA fibers, fusomers with 15-nt dsDNA stem, and fibers with embedded NF-κB decoy samples (1 μM) were aliquoted (5 μL per tube). For lyophilization method, VirTis SP Scientific Benchtop Pro with an Omnitronics freeze dryer was used. The samples were frozen for 3 minutes in liquid nitrogen before lyophilizing at 20.5° C. shelf temperature, a ˜−91° C. condenser, and ˜20 mT orr vacuum overnight. The samples were collected and equilibrated with atmospheric air, then sealed with parafilm. For vacuum concentration method, a CentriV ap micro-IR vacuum concentrator (L abconco) (Speedvac) was used. The samples were centrifuged at 60° C. until fully dry then sealed with parafilm. All dehydrated samples were kept in a heat block for one week at 55° C. As a control, samples were kept in solution at 4° C. and 55° C. for a week. After a week, the dehydrated samples were rehydrated with ddH 20 (5 μL), mixed gently by pipetting, and centrifuged. Rehydrated samples and all control samples were visualized on a native-PAGE (Mini-PROTEAN Tetra system (Bio-Rad)) or kept in the cold room 4° C. until needed. For assessing the stability, an 8% (37.5:1) polyacrylamide native-PAGE was run in running buffer (89 mm tris-borate (pH 8.2) and 2 mM M gCl₂) at 300 V for 20 min in a cold room (4° C.). Total ethidium bromide staining was done for 5 min before imaging the gel using a ChemiDoc MP system (Bio-Rad).

### Temperature Gradient Gel Electrophoresis (TG GE)

To analyze the thermal stability of different fibers, RNA fibers, fusomers with 8, 15, and 30-nt dsDNA stem were assessed via Temperature Gradient Gel Electrophoresis (TGGE). 6% native-PAGE (37.5:1 acrylamide: bis-acrylamide) was prepared using the 15 wells comb. A Mini-PROTEAN Tetra Cell system (Bio-Rad) was used to prepare the gel, then the gel was pre-run at 200 V for 10 min, with running buffer (89 mM TB (pH 8.2), and 2 mM M gCl₂). The samples (1 μM) were mixed with native-PAGE loading buffer (Assembly buffer, 30% glycerol, bromophenol blue, xylene cyanol), (v/v), 1.5 μL of the mixture were loaded per well in the 11 middle wells. The gel was run for 10 min with constant 200 V in running buffer (89 mM TB (pH 8.2), and 2 mM M gCl₂). For the TGGE setup, standard protocol was followed (22). The small plate with the gel was transferred to the TGGE device, and the required temperature range (40-70° C.) of perpendicular settings were used. The TGGE thermal block was run for 20 min at 200 V. Ethidium bromide (EtBr, 0.5 μg/mL) was used to stain the gel for 5 min, then the gel was washed twice with double-deionized water (ddiH₂O). A ChemiDoc MP (Bio-Rad) was used to image the gel. To determine the melting temperature (Tm) value of each sample, the temperature range was divided by the total number of wells loaded minus 2, and the inflection point of the location of the gel bands were used to determine the melting point of each sample.

### Fusomers with Antithrombin Activity

### Prothrombin Time, Activated Partial Thromboplastin Time, and Thrombin Time Assessment

Blood was obtained under FM USP Protocol NP 1378/18. The blood was collected from 10 healthy donors and anti-coagulated with sodium citrate. All the donors agreed with the use of their samples in this research and signed a respective informed consent. A plasma pool was prepared by spinning down the blood in a centrifuge for 10 min at 2500 g and was used within 8 h after collection. 50 μL of NU 172 aptamer decorated fusomers with concentration of 3 μM were added to 450 μL of human plasma in a 1.5 mL Eppendorf tube. NU 172 is a thrombin binding aptamer selected to inhibit coagulation by binding and inactivating thrombin (43, 44, 45). Each tube was incubated at 37° C. for 30 mins. Respective reagents were automatically added to induce the coagulation cascade according to manufacturer protocols. An INSTRUMENTATION LABORATORY ACL TOP 350 CTS Coagulation analyzer, was used to determine the PT, APTT, and TT. PT, APTT and TT assays were conducted using clinical grade instruments and WHO-certified reagents commonly used in the clinic to identify deficiencies in blood coagulation and to monitor the efficacy of anti-thrombotic therapies. Due to the high precision of coagulation instruments (coefficient of variation less than 5%) and the short (in seconds) nature of plasma coagulation, quantitative comparisons of plasma coagulation time between samples, even when statistically significant, does not provide a meaningful estimation of the treatment effect in plasma coagulation.

### Assessment of Fusomers Immunorecognition

Immune reporter cell lines THP-1 dual, human embryonic kidney (HEK)-Lucia RIG-I, HEK-Blue hTLR3, hTLR7, and hTLR9 (InvivoGen) were maintained at standard conditions of 37° C. and 5% CO₂, following InvivoGen's protocols. THP1-Dual cells were plated at 100,000 cells per well in a 96-well Greiner plate. While HEK-Lucia RIG-I and HEK-Blue hTLR 3 and hTLR 7, were seeded at a density of 50,000 cells per well in a 96-well Greiner plate. HEK-Blue hTLR9 cells were seeded at 80,000 cells per well in a 96-well Greiner plate. Immediately after plating, the cells were transfected with positive controls, and 10 nM final concentration of different fibers. Prior transfection, all fiber samples, and positive controls (RNA cubes and 2′,3′-cGA MP only) were incubated with L2K at room temperature for 30 min. For THP1-Dual cells, 1.9 g/mL 2′,3′-cGAMP and 3 μg/mL Pam3CSK4 were used as positive controls for activating the IRF and NF-κB, respectively. A final concentration of 10 nM per well of RNA cube was used as a positive control for HEK-Lucia RIG-I. For HEK-Blue hTLR3 and hTLR 9, 20 μg/mL Poly I: C was used as a positive control. For HEK-Blue hTLR7, 5 μg/mL R 848 was used. Post transfection, the cells were incubated at 37° C. and 5% CO2 for ˜24 hr, then assessed via immune reporter assays and cell viability assay. QUANTI-Blue (InvivoGen) assay was used to assess the secreted embryonic alkaline phosphatase (SEA P) levels in HEK-Blue hTLR3, hTLR7, hTLR9, and THP1-Dual cells. While QUANTI-Luc (InvivoGen) assay was used to assess IRF activation in THP1-Dual and HEK-Lucia RIG-I cells. The manufacturer's guidelines were followed, then a Tecan Spark microplate reader was used to measure absorbance at 638 nm for QUANTI-Blue assay and luminescence (100 ms reading time) for QUANTI-Luc assay. For assessing cell viability post transfection, MTS colorimetric assay (Promega) was done, following manufacturer's protocol. The absorbance was read at 490 nm using the Tecan Spark microplate reader. Each experiment has 3 biological repeats, hence, results were averaged and normalized to untreated cells to obtain fold induction or cell viability.

### Fusomers Uptake Assessment in 3D Organ-on-a-Chip

### OrganoPlate Culture

OrganoReady® Colon Caco-2 plates (Mimetas BV, The Netherlands) were prepared and used for assessing the uptake of fluorescently labelled fibers. The plate contains 64 chips made of Caco-2 tubules seeded against collagen I (21-24). The tubules form a leak-tight barrier that is used to assess the integrity of the barrier of the 3D culture (25). IRD 800 labelled RNA fibers and fusomers were previously assembled and characterized. The labelled RNA fibers and fusomers were complexed with DOTAP DOPE and Lipofectamine™ 2000 Transfection Reagent (L2K) (Thermo Fisher Scientific, USA), and incubated for 30 min at RT, before adding media to reach the final volume. The final concentration tested was 20 nM, 50 nM, and 100 nM. Vehicle control included DOTAP DOPE and L2K, and the negative control was untreated cells. All samples were tested in triplicate. Prior to transfection, all inlets and outlets were aspirated, 50 μL of fresh media was added to all left inlets and outlets, while 50 μL of treatment were added to all right inlets and outlets. Then, the OrganoPlate® was incubated for 24 hr on the OrganoFlow® rocker (Mimetas BV, The Netherlands) at 14°/8 min settings in a 37° C., 5% CO₂ incubator.

### TEER Analysis

To measure the barrier integrity of the Caco-2 tubules, the transepithelial electrical resistance (TEER) values were measured. TEER values provide sensitive measurements of the tightness of the tubules, and insights about the toxicity of the transfected fibers by assessing their barrier induced disruption at the end of the experiment. Hence, TEER values were measure before transfection, and 24 hr post transfection, via OrganoTEER® (Ω*cm²) (Mimetas BV, The Netherlands). In this experiment, the transfected fibers were tested for their barrier disruption effects on the tubules. As a positive control for the barrier disruption, a protein kinase inhibitor (staurosporine (33 nM)) was used as it significantly disrupts the barrier integrity. Before measuring the TEER values, the OrganoPlate® was equilibrated to RT for 30 min, then the TEER is measured at the designated timepoints and recorded for analysis.

### Imaging

24 hr after transfection, the experiment was terminated by fixing and staining the cells for imaging. The cells were washed and fixed for 15 min with 3.7% formaldehyde (Sigma) in HBSS with Calcium and Magnesium (Gibco). Then, the cells were washed twice with PBS (Gibco) for 5 min. Cells were stained immediately, the nucleus staining used NucBlueTM Fixed Cell ReadyProbes™ Reagent (Thermo Fisher, USA), following the manufacturer's protocol. The OrganoPlate® was incubated for 15 min on a RT rocker. After incubation, the cells were washed with PBS for 1 min, and all wells were aspirated. 50 μL of PBS was added to all inlets and outlets including the gel inlets and the observation windows. For imaging the OrganoPlate®, Cytation 5 Multi-Mode Microplate Reader (BioTek, USA) was used, DA PI and CY 5 filters were used at 4X. Fiji was used for images analysis, and uptake quantification.

### Nanopore Analysis

### Nanopore Fabrication

We used high-stress silicon nitride (250 M Pa SiN) membranes supported by a Si chip as substrates for nanopore fabrication, as previously described (23). Nanopore fabrication was carried out using a JEOL 2010F transmission electron microscope operating at 200 kV. Following fabrication, nanopore chips were cleaned in hot piranha (2:1 H₂SO₄/H₂O₂) for 30 minutes, followed by hot deionized water and drying under nitrogen, immediately prior to each experiment. After cleaning, nanopore chips were assembled in a custom flow cell equipped with A g/A gCl electrodes, and a quick-curing silicone elastomer was applied between the chip and the cell to seal the device and thereby reduce the noise by minimizing the chip capacitance. Sample was added to cis (grounded) electrode and positive or negative voltage was applied to trans chamber. Ionic current through the nanopore was measured using either an Axopatch 200B amplifier digitized at 250 kHz sample rates or a Chimera VC100 amplifier (Chimera Instruments LLC) (24) digitized at 4.17 MHz sample rates. Data analysis was carried out using Pyth-lon software (https://github.com/rhenley/Pyth-lon/) for loading, low-pass filtering, and extracting event parameters. Igor Pro (Wavemetrics) was employed for plotting.

### Sample Preparation

Complex solutions of proteins and fibers were made by gently pipetting protein and fibers solutions and then incubating at room temperature for one hour. The complex was stored at 4° C. until used.

### Immunology

### Primary Human Peripheral Blood Mononuclear Cells (PBM Cs) for Analysis of Cytokine Secretion

The blood of three healthy donors was collected and anticoagulated with Lithium-heparin. The anticoagulated blood was mixed 1:1 with PBS at room temperature and layered on top of Ficoll-Paque. Blood samples were centrifuged at 900 g with low acceleration and no brake for 30 mins at room temperature. The monolayer containing the PBM Cs was collected, and 1X Hank's Balanced Salt Solution (HBSS) was added at three times the volume. The solution was centrifuged at 400 g for 10 minutes at room temperature. The washing step was repeated, and the mononuclear cells were resuspended in complete RPM I medium. Cells were then stained using AOPI staining solution for cell counting and plated at 200 k cells per well at a volume of 160 μL in a U bottom plate.

To test the PBMCs with the DNA cubes, RNA cubes, fibers and fusomers, using L2K, a final concentration of 10 nM of NANP per well was added after being incubated with L2K (0.375 μL per well) at room temperature for 30 minutes and then was transfected in duplicate per donor. Samples were diluted in Opti-MEM to bring the volume of each well to 200 μL per well.

The positive controls were added to PBM Cs, LPS (final 20 ng/ml), ODN 2216 (final 5 μg/mL), and PHA-M (final 10 μg/mL). As a negative control, 1X PBS was added to PBM Cs. The supernatants for the positive controls were pooled 1:1:1 to be run on the multiplex plate.

24 hours after transfecting the cells with the nucleic acid nanoparticles, 20 ng of LPS was added to all of the treatments. After spiking the plate with LPS, plates were incubated for 20 h at 37 C and 5% CO₂. Afterward, the plate was spun at 400 g for 5 minutes, and the supernatants were transferred to a new 96-well plate for analysis. The supernatants were tested using a 15-multiplex plate (Quansys) following the manufacturer's protocol. A Quansys ImagePro reader equipped with Q-view software was used to read the multiplex assays wherein cytokine elevation of two-fold or more above the baseline was considered physiologically relevant.

### Gene Silencing

### RNA GFP DS Assembly

The sense and antisense strands were mixed at an equimolar ratio with endotoxin-free water. Once mixed, they were incubated at 95° C. for 2 min. Subsequently, 5X assembly buffer (containing 10 mM Mg2+ and 250 mM K+) was added at 20% of the final volume. The sample was then allowed to equilibrate at room temperature for 20 min before being stored at 4° C.

### Sample Preparation

Prior to all transfections using MDA-MB-231 eGFP cells, the RNA GFP dicer substrates (DS) and GFP fusomers were assembled at a 1 μM concentration. These constructs were then complexed with Lipofectamine 2000 (L2K) and incubated for 30 min at room temperature before transfection. Subsequently, samples were brought up to 50 μl using media to maintain final concentrations of 10 nM or 50 nM as appropriate.

### Transfection and Imaging

MDA-MB-231 eGFP cells were grown and maintained with complete DMEM (consisting of DM EM with 4.5 g/L D-Glucose, L-glutamine, 10% heat-inactivated FBS, 100 μg/mL penicillin, and 100 μg/mL streptomycin) and incubated at 37° C., 5% CO₂. MDA-MB-231 eGFP cells were seeded in a 24-well Greiner plate at ˜40,000 cells per well in a 200 μl volume. The cells were incubated for 24 h at 37° C. with 5% CO₂ before transfection. After the initial incubation, the cells were transfected with GFP fusomer at final concentrations of 10 nM and 50 nM, as well as with the control RNA GFP dicer substrate (DS) at the same concentrations. All treatments were performed in a total volume of 50 μl, bringing the final volume to 250 μl/well.

The cells were incubated at 37° C. with 5% CO₂ for 72 h post-transfection. After the treatments, the media was removed from the cells, and 100 μl of 1X phosphate-buffered saline (1X PBS) was added. The cells were then visualized using the EVOS cell imaging system.

Fusomers with Antibacterial Activity

For bacterial growth assay, K12 E. coli was grown from single colonies in LB at 37° C. overnight in a GeneM ate Incubated Shaker Mini with 200 rpm constant shaking. Bacteria were diluted to an optical density at 600 nm (OD600) of 0.02, with LB. In a sterile 96-well black-walled plate, 50 μL of diluted bacteria were added. Samples were added with LB to bring the final volume to 100 μL in each well, 1, 2, 4, 6 UM of Fusomers AgNCs, and C12 AgNCs, final bacteria at an OD 600 of 0.01. Silver control was added replicating the highest concentration of silver in the DNA-AgNCs samples (6 μM nucleic acid). For the positive control, Carbenicillin was added for a final concentration of 10 μg/mL. The fusomer and C12 hairpin were used as DNA controls at 1 μM. A 10 mL solution of 20% ethanol, 0.05% Triton-X 100 was used to hydrophobically treat the lid of the plate, the solution was poured into the lid for 30 seconds, and the excess was poured off, the lid was allowed to dry upright in the biosafety cabinet for 30 min (25). The plate was covered with the lid and sealed with parafilm to prevent excess evaporation. Tecan Spark microwell plate reader was used to obtain optical density measurements. The plate was incubated in the plate reader at 37° C., and shaken for 30 seconds before each OD₆₀₀ measurement occurring every 15 minutes over 24 hr. A total of three biological repeats, with three technical repeats for each experiment, was performed. GraphPad Prism 9 was used to calculate and plot the growth curves as the average OD₆₀₀ at each timepoint, with a standard error of the mean (SEM) of each measurement (16).

For mammalian cell viability assays, HEK 293-FT cells growing in DM EM, 2 mM L-glutamine, 1% PenStrep, and 10% heat inactivated FBS, were plated in a 96-well plate at a cell density of 40,000 cells/well. After plating, cells were incubated at 37° C., 5% CO₂ for 24 hr before treatment. Fusomer AgNCs, C12 A gN Cs, silver control, fusomer, C12 hairpin, and 1X Assembly buffer were used for cell treatment, media was added to bring the final volume to 100 μL per well. The cells were incubated with the treatments for 24 hr, then 20 μL of CellTiter 96® A Queous One Solution Cell Proliferation Assay (MTS) were added to each well. The plate was incubated for 75 min before measuring absorbance at 490 nm using a Tecan Spark microplate reader (17). A total of three biological repeats, with three technical repeats for each experiment, was performed. GraphPad Prism 9 was used for data analysis and plotting.

For Excitation Emission Matrix (EEM) analysis Fusomers AgNCs and C12 AgNCs were synthesized at 10 uM. A fter overnight incubation, 100 ul of each sample was placed into a 96-well black-walled plate and the initial measurement was taken. The Tecan Spark plate reader measured the intensity at emission wavelengths of 350-700 nanometers after being excited with wavelengths of 400-850 nanometers, with a bandwidth of 5 nm and step size of 5 nm. The gain was manually set at 150. The data was plotted as a heat map using GraphPad Prism.

### Results

### Design of RNA-DNA Fusomers

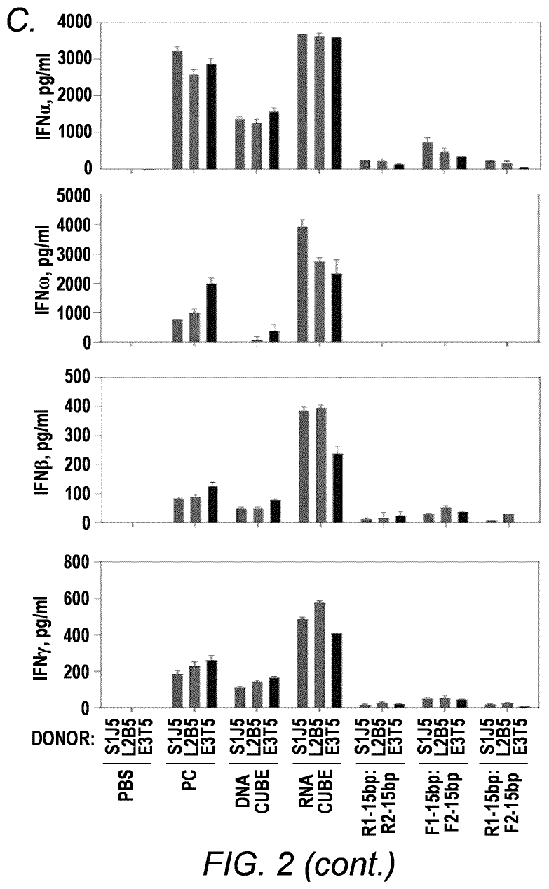

The rational of fusing RNA and DNA oligonucleotides in one sequence, folding into fibrous structures was primarily assessed in human PBMCs for their immunostimulatory response. Here in, we tested various nucleic acid nanoparticles; 3D cubes (DNA and RNA cubes) and 1D fibers (RNA-RNA, RNA-fusomer, and fusomer-fusomer) in human PBM Cs using multiplex ELISA to measure Type I and III interferons; IFNα, IFNβ, IFNω, and IFNλ (FIG. 2). The positive control used is ODN 2216. The results demonstrate that RNA cubes showed the highest immunostimulatory response where all the interferons spiked up, as compared to the response shown from the activation by the positive control. These results agree with our previous findings (3). Where DNA cubes showed lower immunostimulatory response than the RNA cubes and the positive control. While the fibers showed significantly low to zero immunostimulatory response. Where (RNA-fusomers) fibers showed slightly higher response as compared to (RNA-RNA) fibers and (fusomer-fusomer) fibers, however the slight immune response is almost negligible as compared to the response from cubes. These results demonstrated that changing the composition of fibers did not stimulate immune response, hence the immunoquiescent platform could be functionalized for various applications in mammalian cells.

### Changes in Architectural Parameters of Fusomers

### AFM Imaging and Image Analysis

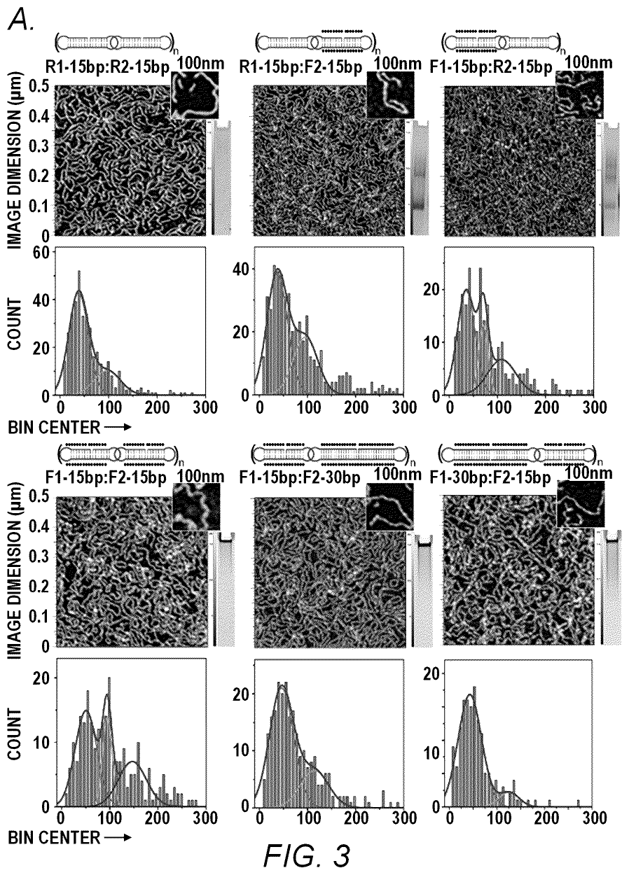

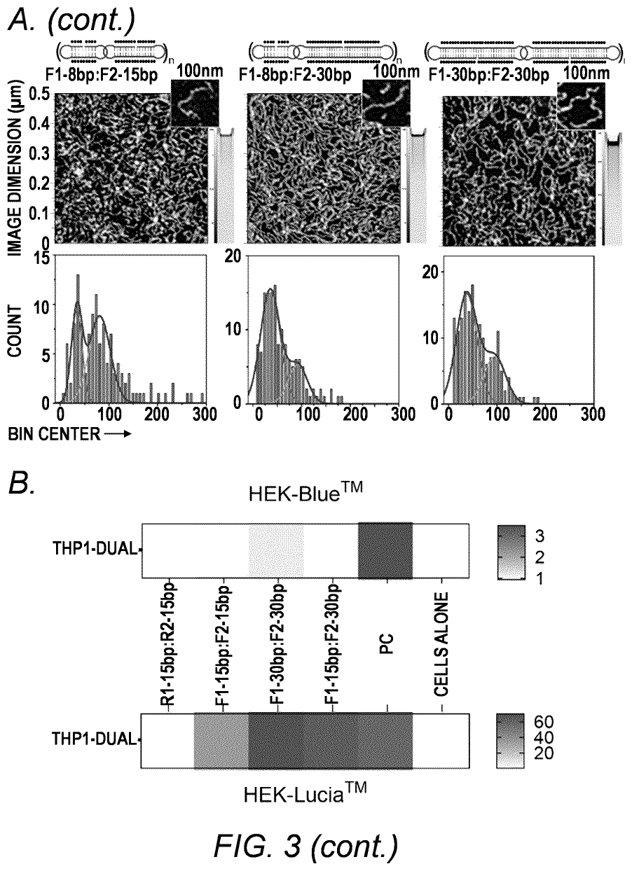

A total of 9 samples of various fusomer composition were imaged using AFM topography imaging (FIG. 3). We further assessed their morphological and mechanical properties through image analysis (FIG. 3, panel A). AFM imaging is a perfect technique for visualizing and precisely extracting the structural and mechanical properties of fibrillar nanostructures (26). Topological statistical analysis of structural conformations measured with AFM imaging, in many cases, shows a perfect agreement with the mechanical properties of nanostructured objects (27-30). For the studied RNA/DNA designs, AFM imaging revealed fibrous nanostructures of continuous nature. While the nanostructures were identified as continuous, they also appeared curved. Curvature represents a central morphological feature of many biological structures (31). For materials of a linear morphology, such as the RNA/DNA polymer chains we studied here, it could indicate the mechanical robustness of the continuous morphology and internal structural organization (32, 33). We noticed that the appearance of the nanostructures varies as we varied the composition of constituent building blocks. We aimed to evaluate the mechanical properties of the nanostructures, and for that, we thoroughly analyzed the images. The primary parameter we analyzed is the curvature of the fibrous nanostructures, measured at multiple points along each individual fiber. The curvature, represented by the values of κ at a point on the line, can be intuitively defined as the inverse of the radius of an “oscillating circle” fitted to the arc of the curve at each point on the fiber (34). This curvature reflects the bending rigidity of a nanostructure at each measured point. The larger the value of κ, the more curved the nanostructure is at that point. Therefore, this parameter reports on mechanical properties of the samples and how these properties are influenced by the different composition of the constituent building blocks. The values of curvature, κ, were collected, averaged per each fiber structure, and organized into statistical histograms (FIG. 3, panel A). The resulting statistical histograms reveal a wide range of curvature values, indicating the heterogeneous characteristics of the nanostructures and the influence of compositional blocks on both the structural order and the macroscopic mechanical properties of these nanostructures.

Without wishing to be bound by any particular theory, we expected two major contributing factors to flexibility of the studied fibers: 1) the composition of the polymer, distinguishing between pure RNA (R-mer) and a blend of RNA/DNA sequences (Fusomer), and 2) the density of kissing loops along the polymer chain. We begin with exploring the impact of polymer composition by comparing fibers composed solely of RNA monomers with those incorporating Fusomers. FIG. 3, panel A illustrates this relationship. It is evident that substituting both RNA monomers with Fusomers (F15-F15) significantly enhances fiber flexibility, as indicated by the broader distribution of κ-values in the histograms. The average value of curvature, κ, for the R15-R15 sample is 62 μm-1 significantly lower than the 125 μm-1 observed for the F15-F15 sample. Introducing just one Fusomer (F15) into the chain—either as the first (F15-R15, κ=81 μm-1) or second (R15-F15, κ=92 μm-1) monomer—results in intermediate curvature values, as expected. These findings suggest that substituting RNA with Fusomer enhances the overall flexibility of the resulting polymer chain as indicated by increased kappa, κ, values.

Further analysis involved fitting Gaussian functions to the apparent distribution of curvature values, providing insights into the relative prevalence of straight versus flexible, curved regions of the fibers. The first Gaussian peak corresponds to straighter nanostructures, while subsequent peaks at higher κ-values represent contributions from more curved, more flexible chains. As depicted in FIG. 3, panel A, the presence of flexible nanostructures becomes more pronounced with the introduction of Fusomers, especially when both RNA monomers are replaced by Fusomers.

Next, we explored the influence of kissing loop density on the flexibility of the nanostructures. Given that kissing loops act as hinges, their higher densities per unit length are anticipated to reduce fiber stiffness. The results, presented in FIG. 3, panel A demonstrate this effect. Starting with a composition of F15-F15 we replaced the second monomer with F30. This replacement significantly enhanced stiffness and reduced kappa to 82.9 μm-1. Similarly, F30-F15 composition results in stiffer nanostructures with kappa being=58.0 μm-1. Further exchange of the first F30 with F8 results in loosing fibrillar stiffness (F8-F15, κ=86.7 μm-1).

The stiffness recovers when second monomer F15 is replaced with F30 (F8-F30, κ=57 μm-1) and swapping F8 for F 30 (F30-F30) results in a larger kappa of 83.0 μm-1. In general, the trend aligns with expectations, showing increased flexibility as longer monomers are substituted with shorter ones and larger curvature values are evident in the statistical distribution. However, the impact is less pronounced when compared to the polymer composition trend (FIG. 2).

In summary, the analysis of the structural features, the physical, and mechanical properties of the studied nucleic acid-based nanostructures is essential for our understanding of their biological role and achieving their successful applications in nanotechnology and material science. Using A FM imaging and image analysis we were able to delineate the contributions of the constituent building blocks to overall mechanical properties of NA-based nanofiber structures. It appears that the bending rigidity is mainly governed by the polymer composition rather than kissing loop density in the studied nanostructures.

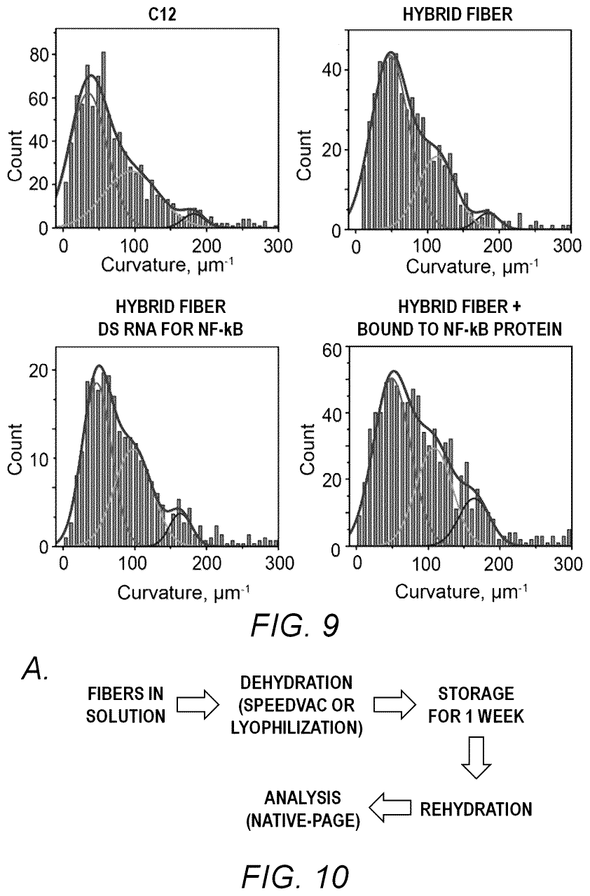

Finally, AFM image analysis of functionalized fusomers was done. Results show that functionalized fusomers exhibited similar topological features to the unmodified ones, with linear nanostructures showing curvature that indicates flexibility, which is dependent on their composition. Generally, modifications lead to increased flexibility, as demonstrated by a broader distribution of κ (kappa) values in the histograms, as shown in FIG. 9. The presence of more flexible fibers is further evidenced by a larger contribution of Gaussians with higher κ values. The specific κ values obtained are as follows: κ=78.6 μm⁻¹ for F-C12, κ=82.1 μm⁻¹ for the Hybrid fiber, κ=91.7 μm-1 for DS RNA for NF-κB, and κ=107 μm-1 for the Hybrid fiber with NF-κB decoy bound to NF-κB protein. Interestingly, the binding of the protein increases fiber flexibility, as indicated by a significant presence of higher k values in the distribution.

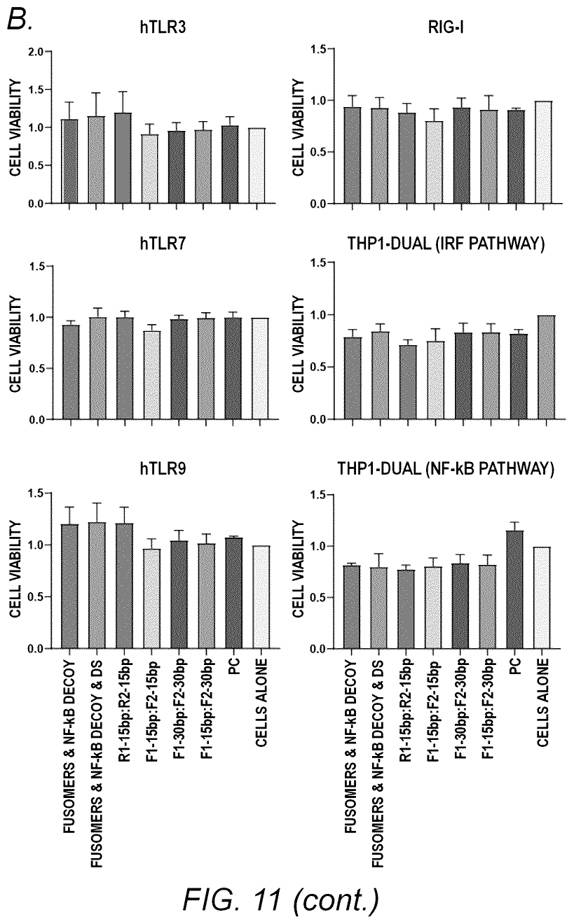

The effect of the tunable mechanical properties by varying structural features of linear fibers was assessed on THP-1 dual reporter cells, which are engineered with reporter genes driven by crucial immune signaling pathway, NF-κB and IRF. The aim of this experiment is to investigate if the length and composition variation would have immunostimulatory effects by activating the NF-κB or IRF pathways or both. FIG. 3, panel B demonstrate that there is minimum activation to the NF-κB pathway upon treatment with (R15-R15) fibers, (F15-F15), (F30-F30), and (F15-F30) fusomers, the results are consistent with previous work reported by our lab (38). However, IRF pathway activation was shown when treating with (F15-F15), (F30-F30), and (F15-F30) fusomers, but not with (R15-R15) fibers, due to activation of cGAS-STING pathway by DNA which is consistent with previous findings (3, 38, 39). W herein, (F30-F30) had a higher immunostimulatory effect as compared to (F15-F30) indicating that the cGAS-STING pathway is activated in a length-dependent fashion (40). Furthermore, the panel of fibers were also assessed for their immune recognition in HEK-Blue hTLR3, HEK-Blue hTLR7, HEK-Blue hTLR9, HEK-Lucia RIG-I, which are engineered to express PRRs specific for the recognition of nucleic acid. The data shown in FIG. 11, panel A revealed that there is minimum activation of these reporter cells, indicating that these PRRs are not activated, nor involved in the recognition of the RNA fibers or fusomers, in agreement with data of the PBM Cs cultures discussed above, and in alignment with previous results (38). Additionally, the viability of the reporter cells treated with the panel of fibers was assessed, and the results indicate that there is no significant decrease in the cell viability (FIG. 11, panel B).

Collectively, this knowledge allows the modulation of the fiber composition, length and functionalization, based on the flexibility and the immunorecognition level required for the desired biomedical application.

### Dehydration and Stability

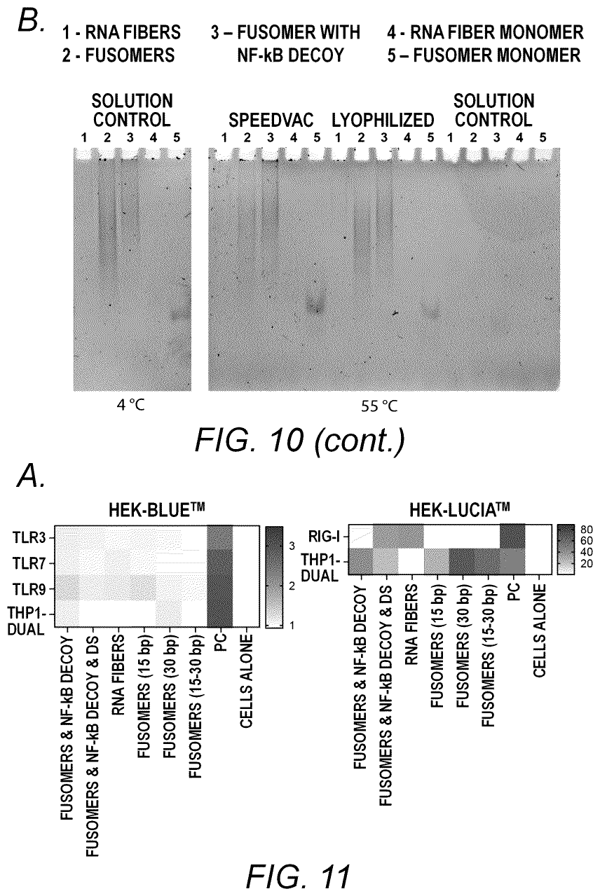

The thermal stability of fibers and fusomers is essential for maintenance of their structural integrity during storage and shipping. Hence, the thermal stability of a panel of fibers (R15-R15), (F15-F15), and fusomer functionalized with NF-κB decoy is investigated. The panel of fibers is dehydrated via 2 methods; vacuum concentration and lyophilization, then stored at 50° C. for a week, after that the samples are rehydrated and run on a Native-PAGE along with controls as shown in FIG. 10, panel A. The Native-PAGE shows that dehydrating samples via vacuum concentration and lyophilization preserved the integrity of the fibers when stored at high temperature for a week, as compared to the solution control that was stored at the same temperature (FIG. 10, panel B). These results align with previous findings reported by our lab (10), indicating that thermal stability of fibers and fusomers will not impede their storage or delivery at ambient temperatures while maintaining their structural integrity.

### Fusomers Uptake Assessment in 3D Organ-on-a-Chip

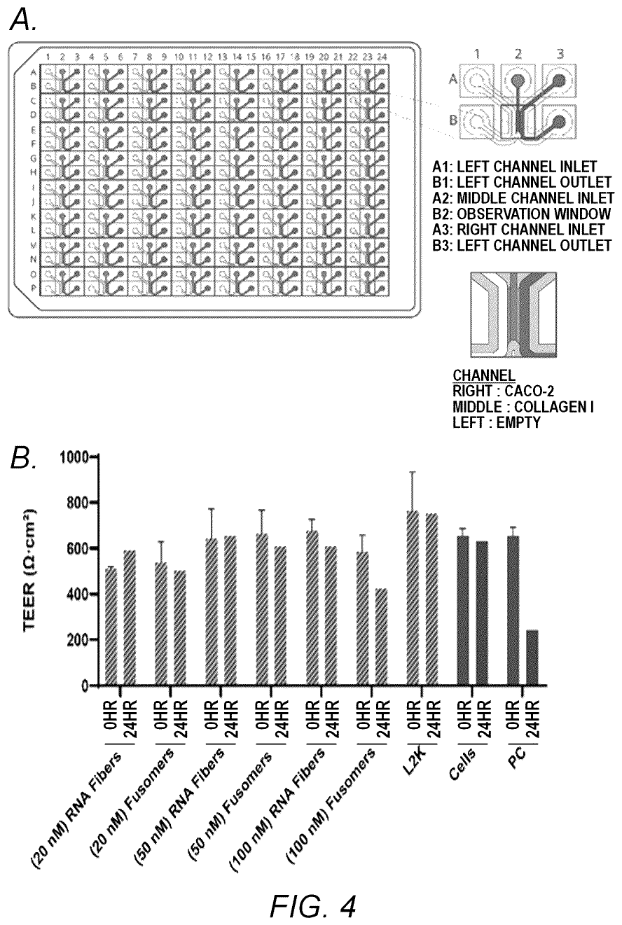

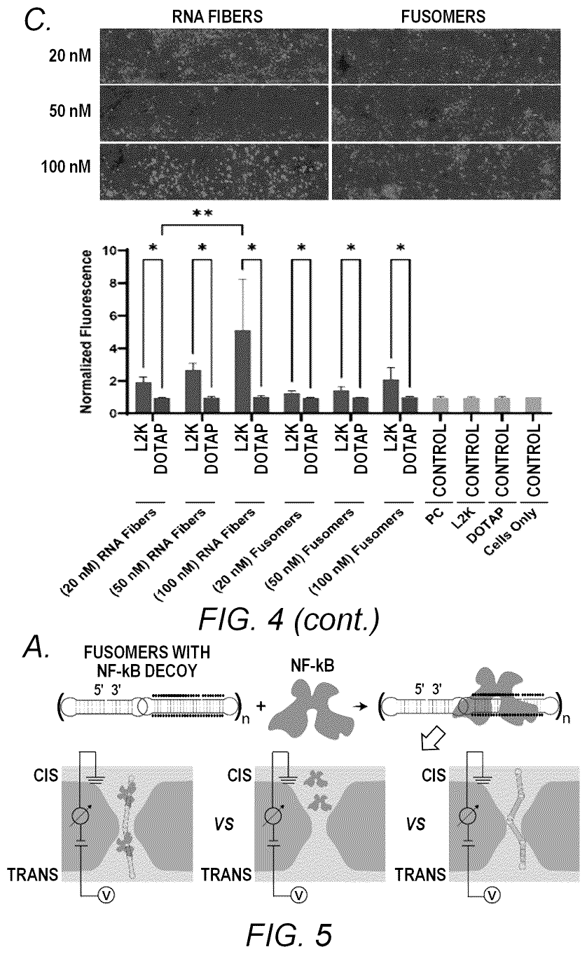

For relative uptake of fibers in 3D culture, a 3-lane 64 OrganoPlate® was seeded with Caco-2 culture forming tubules. The plate is a standard 384-well plate, comprising 64 chips, each chip is made of 2×3 block of wells. Every chip includes a right channel inlet and outlet (A3, B3), left channel inlet and outlet (A1, B1), gel channel (A2), and an observation window (B2) showing the intersection of the three channels for imaging (FIG. 4, panel A). To prepare the Caco-2 OrganoPlate®, the gel channel (A2) is seeded with collagen-I and incubated overnight, Caco-2 cells are then seeded into the right channel inlet (B3), and incubated to attach to the gel matrix, then media is added to all right and left channels. The plate is then placed on the OrganoFlow® rocker at 14°/8 min settings in a 37° C., 5% CO₂ incubator, upon perfusion the Caco-2 cells form a 3D tubule in the right channel (FIG. 4, panel A). Prior to transfection, all chips passed minimum TEER requirements and QC, then the fibers were complexed with the respective carrier and then transfected to the Caco-2 tubules to measure their relative uptake and their effect on the barrier integrity after 24 hr.

FIG. 4, panel B shows the TEER measurements highlighting that the barrier integrity of the Caco-2 tubules was not disrupted by any of the treatments or the vehicle controls, as compared to the positive control after 24 hr of transfection. This indicates that the fibers and vehicles are not cytotoxic.

As shown in (FIG. 4, panel C), the fluorescence imaging of the labelled fibers show that the uptake of fibers was concentration dependent. In addition, the RNA fibers and fusomers complexed with L2K had better uptake when compared to RNA fibers and fusomers complexed with DOTAP DOPE. Where, the carrier dependency is obvious with RNA fibers and fusomers complexed with L2K, showing uptake even at the lowest transfection concentration (20 nM), as opposed to those complexed with DOTAP DOPE at the same concentration. Uptake quantification was processed on Fiji, and two-way ANOVA (*p<0.05, ***p<0.001) was performed to determine significance. The graph shows that the relative uptake of RNA fibers complexed with L2K is higher than the relative uptake of fusomers complexed with L2K.

### Fusomers with Antithrombin Activity

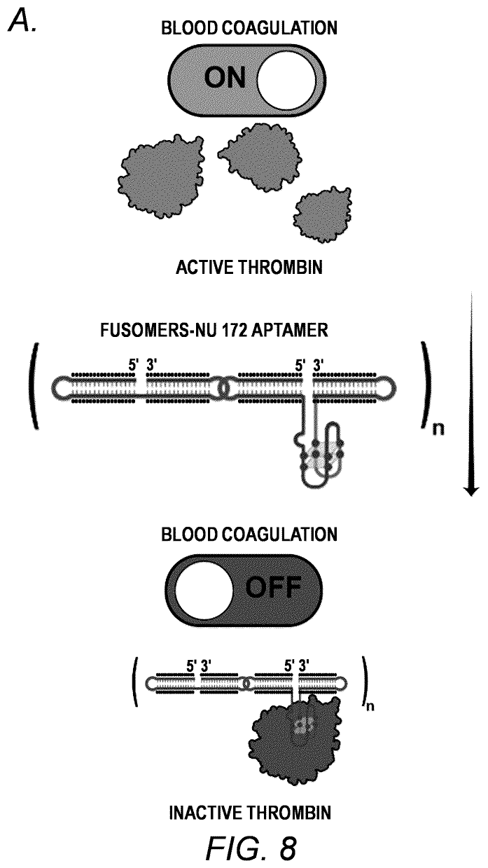

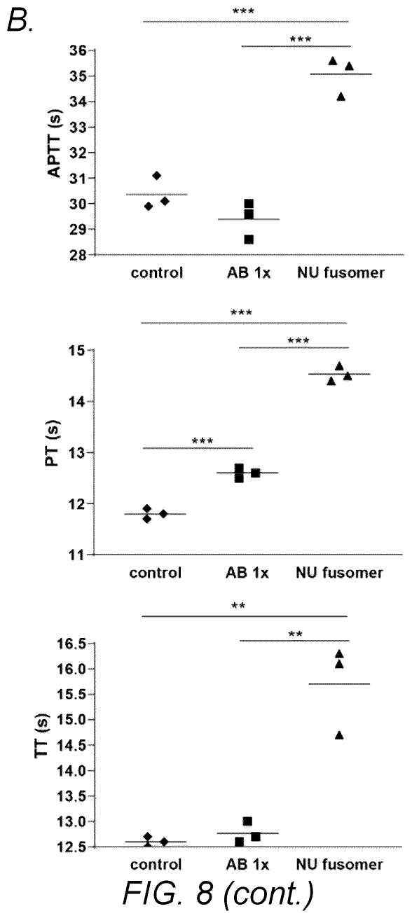

The NU 172 aptamer is known for its anticoagulant activity, however due to its short half-live its biomedical application is hindered. In this study, NU 172 aptamer was selected as a fusomer functionality, as fusomers would provide enhanced stability, increased local concentration, and prolonged coagulation times (FIG. 8, panel A). The Prothrombin Time (PT), Activated Partial Thromboplastin Time (APTT), and Thrombin Time (TT) tests are standard tests used in clinical practice to assess coagulation pathways in human plasma. Activated partial thromboplastin time (APTT) evaluates the functionality of the intrinsic pathway, prothrombin time (PT) assesses the extrinsic pathway, and thrombin time (TT) measures the activity of the common pathway. These tests provide a comprehensive evaluation of the coagulation system, assisting in the diagnosis of bleeding disorders, evaluating hemostatic balance, and monitoring anticoagulant therapy.

To evaluate the anticoagulant potential of NU fusomers, we performed APTT, PT, and TT tests using blood of healthy donors. Coagulation assays were conducted according to current clinical standards utilizing World Health Organization (WHO) certified human plasma as controls and WHO-qualified plasma coagulation reagents with known time limits for normal plasma coagulation. These standards qualified any time measurements below 11.5, 29, and 15.8 s as normal times for PT, APTT, and TT assays, respectively. Coagulation times above these limits were considered as prolongation. The coagulation times in all three assays were significantly prolongated by NU fusomers compared to controls (FIG. 8, panel B).

### Gene Silencing

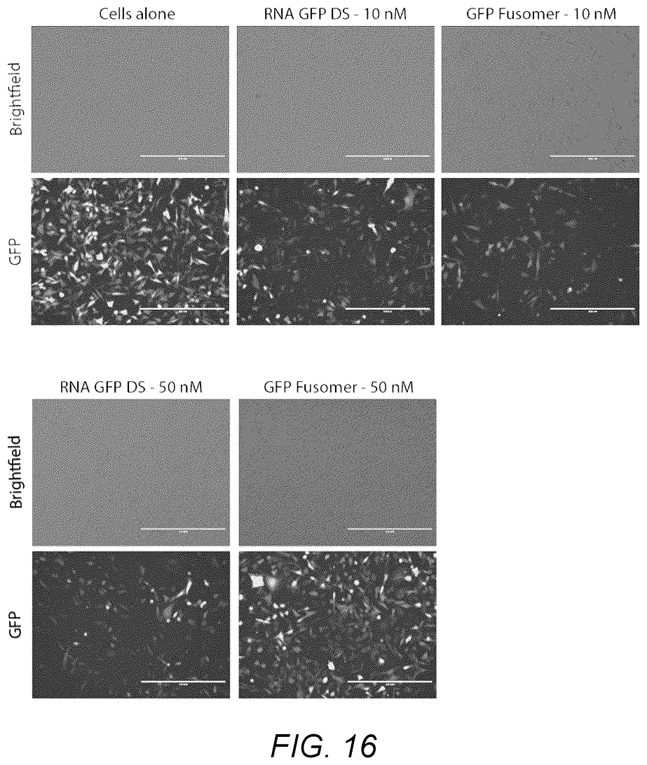

A fluorescent microscope was used to qualitatively assess the silencing treatments on MDA-MB-231 eGFP cells. In FIG. 16, the downregulation of green fluorescent protein (GFP) is visible with both the fusomers and the RNA GFP dicer substrate (DS) compared to the untreated cells.

### Fusomers for Protein Binding (Nanopore Biosensing)

### Nanopore Fabrication and Measurements

Different Fibers through Nanopore

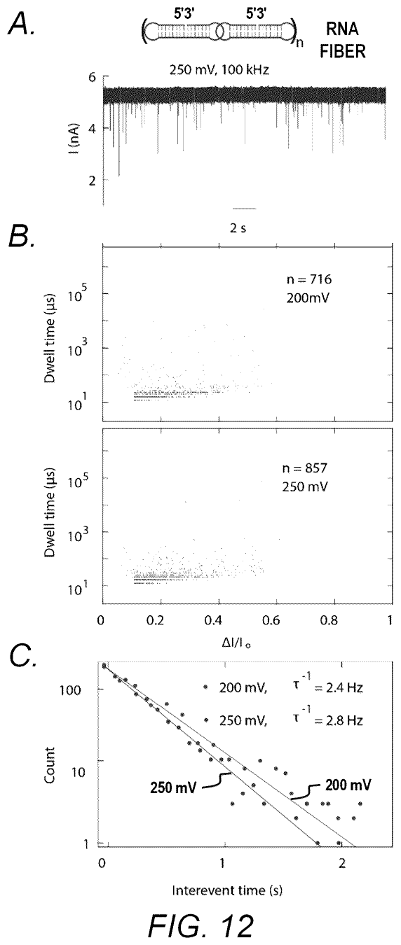

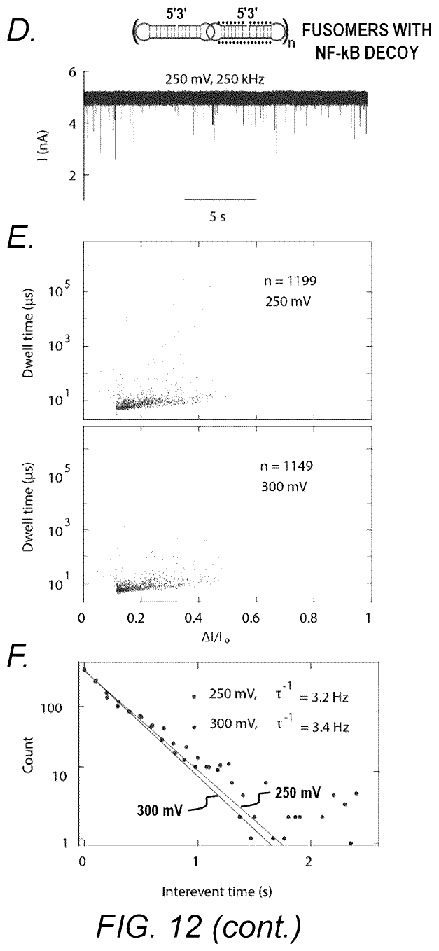

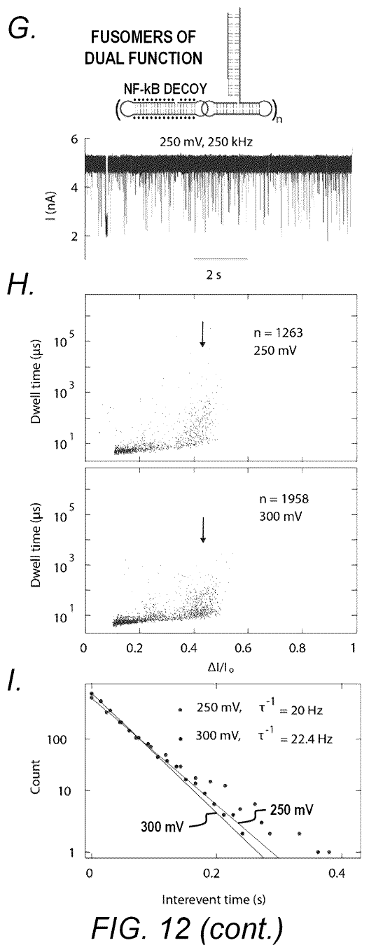

SiNx nanopores were used to characterize RNA fiber, hybrid fiber with NF-κB decoy (unbranched), and hybrid fiber with NF-κB decoy and DS RNA (branched) in 1M KCl, 10 mM HEPES, 2 mM M gCl₂ at pH 7.5. Two different nanopores with similar size were used (size estimated based on the pore conductance), one to characterize the RNA fibers and another to characterize unbranched and branched hybrid fibers. RNA fiber while passing through ˜5.5nm produced fast events with broad distribution in current blockades (FIG. 12, panel A and FIG. 12, panel B) as shown in previous study (35). Interevent time distributions were fit to exponential decay functions to extract the mean capture rates. Capture rates increased with voltage, as shown in FIG. 12, panel C. A similar behavior was observed in the unbranched hybrid fibers (FIG. 12, panels D-F). The branched hybrid fiber, unlike RNA fiber and unbranched hybrid fiber, produced two distinct populations: one similar to the unbranched hybrid fiber and another with deeper and longer events. This observation is similar to a previous study for unbranched and branched RNA fibers (FIG. 12, panel H and FIG. 12, panel I) (35). This data (FIG. 12, panel H) also shows the shift of event population towards lower dwell time region when voltage was increased from 250 mV to 300 mV, which suggests that the fibers translocate through the nanopore. The capture rate for branched hybrid fiber (FIG. 12, panel I) was greater than that for unbranched hybrid fiber for the same concentration, suggesting that the branched fiber was easily detectable using our high-bandwidth electronics.

Characterization of protein bound fibers with nanopore

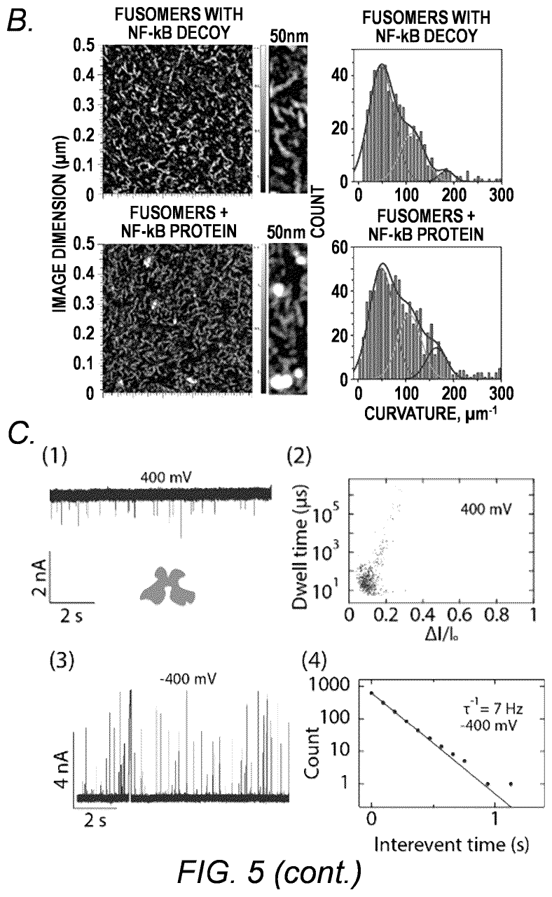

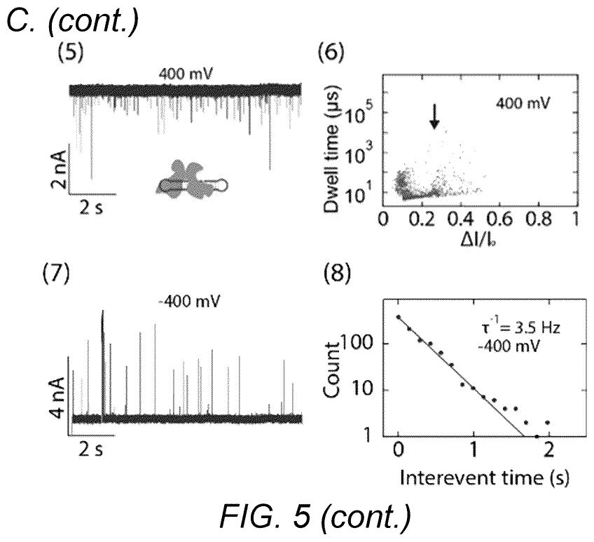

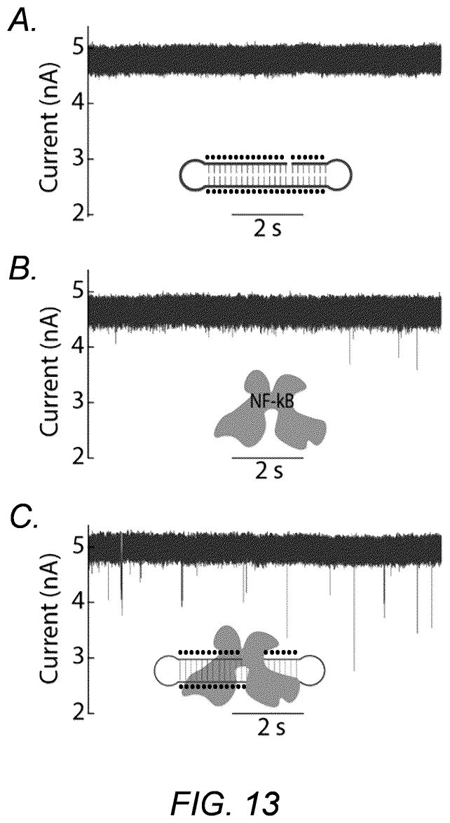

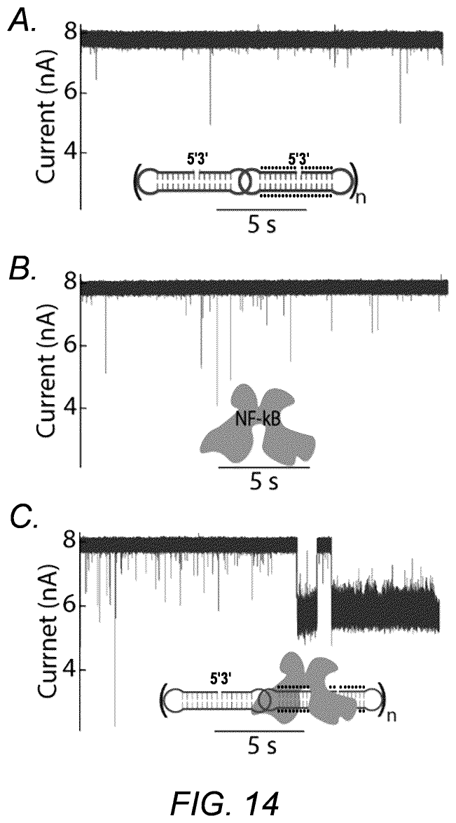

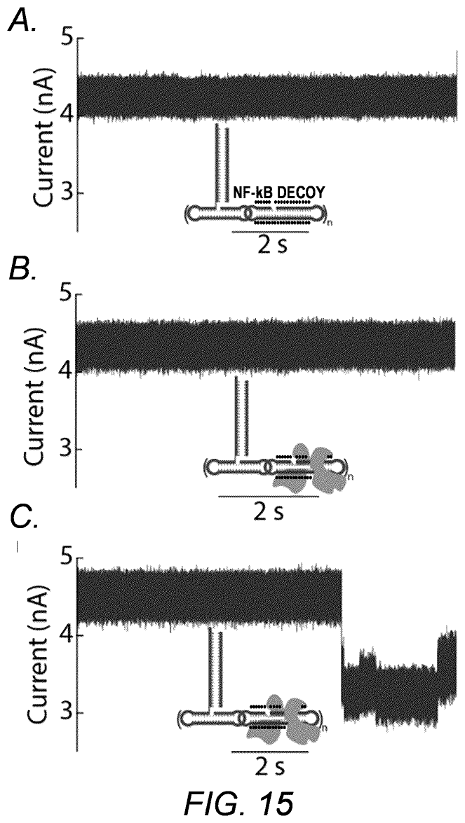

In addition, fusomers functionalized with NF-κB decoys are used as molecular sensing probe of NF-κB protein via nanopores analysis as shown in FIG. 5, panel A (36, 37). The current trace recorded at 400 mV for the NF-κB protein in 1M KCl, 10 mM HEPES, 2 mM MgCl₂ at pH 7.5 with a ˜5.2 nm pore is shown in FIG. 5, panel C, graph (1). The protein is slightly negatively charged (−4.61) in folded state at pH 7.5 calculated by PROPKA (38, 39). A weakly negatively charged protein experiences a weak electrophoretic force supporting translocation and a strong electroosmotic force opposing translocation; therefore, shallow events were observed at positive voltages as shown in FIG. 5, panel C, graphs (1) and (2). At −400 mV, a strong electroosmotic force, which supports translocation, overcomes the weak electrophoretic force, thereby allowing the translocation. Consequently, deeper events are generated when a negative voltage is applied as shown in FIG. 5, panel C, graph (3). In FIG. 5, panel C, graph (4) shows the histogram of interevent time, exponential fit of which gives the capture rate. The complex was prepared by mixing protein and the duplex monomer in 1:2 ratio. When tested at 400 mV, the complex produced a population of deeper events, while such a population was absent for the protein. The current trace recorded at 400 mV for the complex with another pore also showed the deeper events (FIG. 5, panel C, graphs (5) and (6)), which were absent in the current trace of the monomer and protein (FIG. 13). This indicates the successful binding of the monomer to the NF-κB protein. The binding increased the negative charge of the complex, consequently increasing the strength of the electrophoretic force that facilitated translocation, leading to the production of deeper events. Events similar to those of free proteins were observed at −400 mV (FIG. 5, panel C, graph (7)), although with half the capture rate (FIG. 5, panel C, graph (6)), which also indicates that some proteins are bound to the monomer. The concentration of the protein in each sample was 71 nM. Protein bound hybrid fiber with NF-κB decoy showed clogging-like events which were absent for the protein and fiber (FIG. 14). Similar event was observed for protein bound hybrid fiber with NF-κB decoy and DS RNA for the sample prepared by mixing 30% of protein and 70% of fiber (FIG. 15). Those events may indicate the successful binding of the protein with the fibers. The binding is supported by AFM imaging, where the fusomer with NF-κB decoy is shown in free state and NF-κB protein-bound state in FIG. 5, panel B.

### Fusomers for Downregulation of NF-κB

### Primary Human Peripheral Blood Mononuclear Cells (PBMCs) for Analysis of Cytokine Secretion in Response to Fusomers Functionalized to Down-Regulate NF-κB

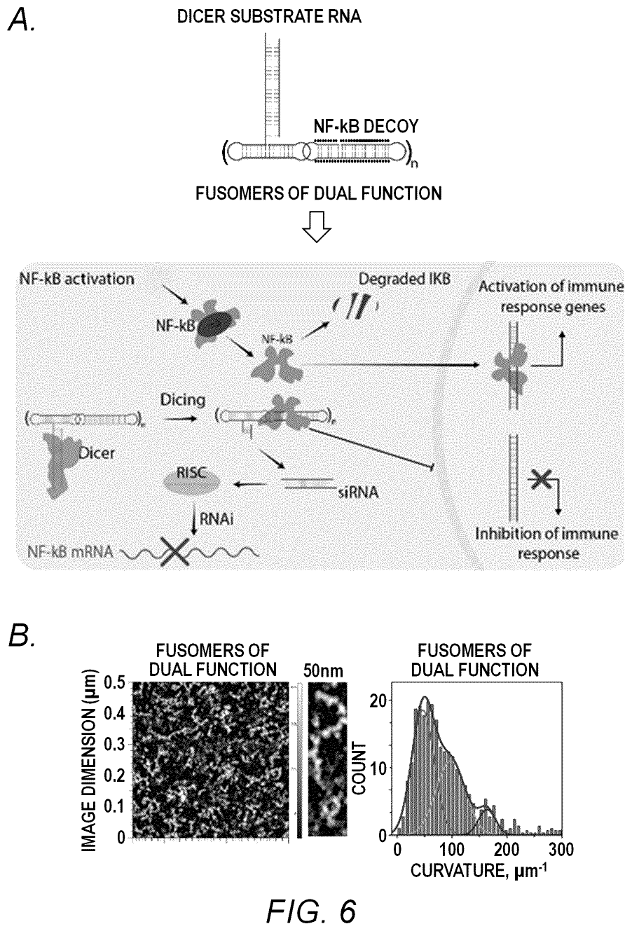

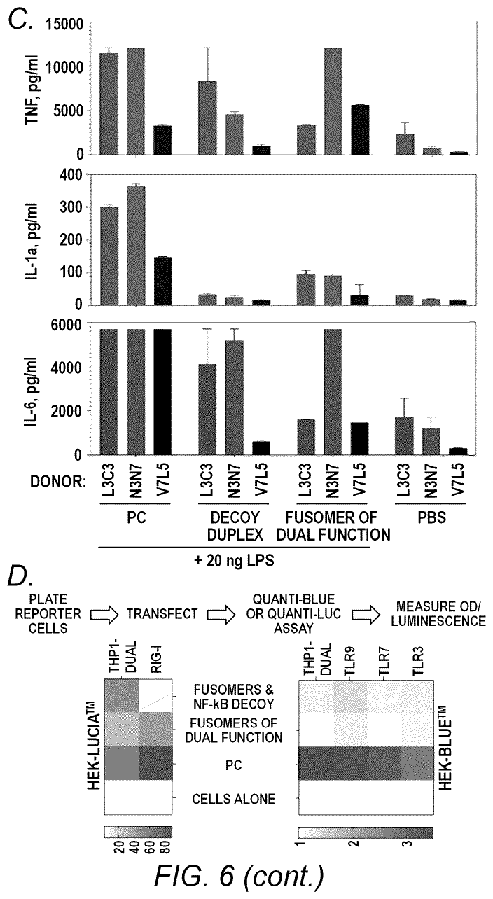

Downregulation of inflammation is achieved by functionalizing fusomers for delivery of NF-κB decoy oligonucleotides which are designed to mimic the KB consensus sequence recognized by NF-κB ultimately blocking its function by preventing its translocation to nucleus (7, 40). Fusomers are also functionalized with siRNAs against NF-κB (6, 41). Consequently, fusomers effectively target the production and activation of already produced NF-κB and inhibit inflammatory signaling pathways through multiple pathways, to meet the therapeutic goals. FIG. 6, panel A demonstrates the design of dual function fusomers, and their activity in the cell for NF-κB downregulation. Dual function fusomers were further characterized by A FM imaging (FIG. 6, panel B).

Dual function fusomers were tested for their NF-κB downregulation activity response in human PBM Cs using a multiplex panel that included Type I and III interferons, as well as TNFa, IL-1a, and IL-6. The positive control was made by combining lipopolysaccharide (LPS), ODN 2216, and phytohemagglutinin (PHA-M). Different control constructs were tested to assess how each aspect contributed to immune responses from the PBMCs.

Tumor necrosis factor alpha (TNFa), Interleukin-1a (IL-1a), and Interleukin-6 (IL-6) are proinflammatory cytokines that were used to assess whether the fusomers and each portion of the fusomer could reduce the immune response after being stimulated with LPS. These cytokines are biomarkers of LPS-mediated activation of PBM Cs which depends on NF-κB activation. FIG. 6, panel C shows the resulting immune responses in TNFa, IL-1a, and IL-6. The decoy duplex can maintain a low immune response in IL-1a, as is the fusomer of dual action. However, IL-6 is stimulated by all samples.

Fusomers of dual function were further characterized along with fusomers with NF-κB for their immune recognition. Reporter cell lines HEK-Blue hTLR3, HEK-Blue hTLR7, HEK-Blue hTLR9, HEK-Lucia RIG-I, and THP1-Dual were used to investigate the recognition of these fusomers by the specific PRRs. FIG. 6, panel D shows that there is no significant activation of NF-κB pathway in the hTLR and THP1-Dual reporter cells. However, there was activation of the IRF pathway in both RIG-I and THP1-Dual cells. The analysis showed that dual function fusomer had higher activation of IRF pathway in RIG-I reporter cells as compared to THP1-Dual reporter cells, indicating that the branched RNA moiety in the dual function fusomer might have played a role in the RIG-I activation. Finally, the cell viability of these reporter cells was assessed upon treatment with the fusomers, and results in FIG. 11, panel B show no significant reduction in cell viability.

### Fusomers with Antibacterial Activity

### Bacterial Growth Inhibition And Mammalian Cell Viability Assays

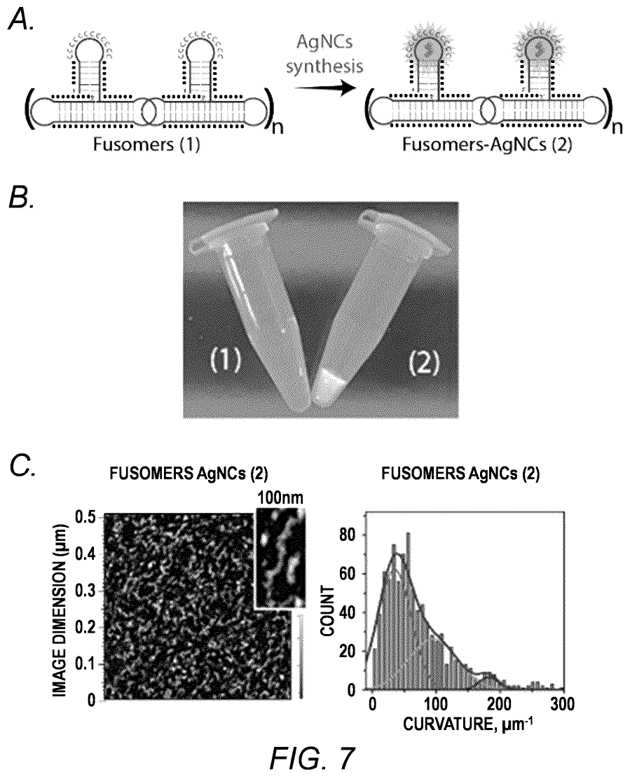

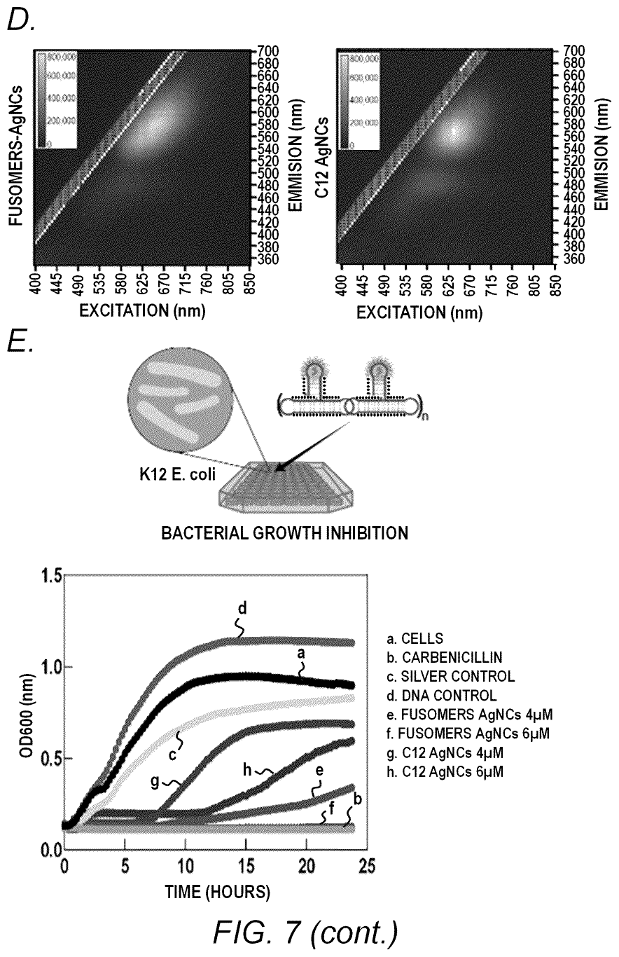

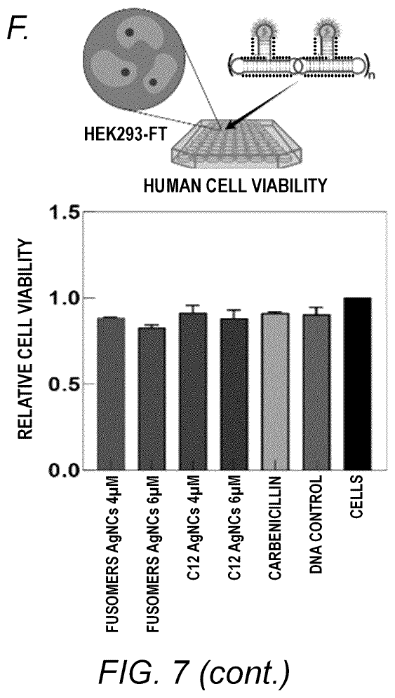

DNA-templated AgNCs are known to have bacteriostatic properties, inhibiting the growth of bacteria over many hours, depending on the concentration of the DNA (16, 17). In this study, the DNA-templated A gN Cs with a 12-cytosine (C12) hairpin loop were used freely and attached to the fusomers, forming Fusomer AgN Cs that fluoresce (FIG. 7, panels A and B). The structure of the fusomers with C12 hairpin is shown in the AFM topography image (FIG. 7, panel C). The Fusomer AgN Cs, with nucleic acid templates of 4 uM, inhibited growth of K-12 E. coli for the same amount of time as free C12 AgNCs at 6 uM as shown in FIG. 7, panel E. This suggests that the fusomer structure increases the local concentration of silver in the hairpin loops attached, which better inhibits the growth of bacteria. Additionally, the Fusomer AgNCs at 6 uM inhibit growth for the entire 24 hr time. Fusomer AgNCs are promising to be used as antibacterial agents; while the ability to inhibit bacterial cell growth was explored, more experiments are needed fully understand the bactericidal abilities and the addition of targeting moieties can be explored to further increase the local concentration of silver. The Fusomer A gN Cs were then tested for human cell cytotoxicity on HEK 293-FT cells. The results show that Fusomer AgN Cs at 4 uM and 6 uM were not cytotoxic, as their relative cell viability is not significantly different than untreated cells, and DNA only controls as shown in FIG. 7, panel F.

### EEM Studies

The Excitation-Emission Matrix (EEM) of each AgNCs sample was used to quantify the fluorescence over a range of excitation and emission wavelengths (FIG. 7, panel D). C12 A gNCs have an emission peak at 645 nanometers when excited with 570 nanometer light. This peak concurs with the visible orange-red color. Additionally, the Fusomers AgNCs have an emission peak at 655 nanometers when excited with 580 nanometer light. A gain, the peak concours with the visible color. The Fusomers A gNCs have a broad, oval-shaped peak, which is different from the small circular peak from the C12 AgNCs. Finally, the C12 AgNCs are more intense than the Fusomers AgNCs, which is interesting because visually the Fusomers AgNCs are visually to be brighter. The reading may be from the localization of the emission wavelengths, rather than a more oblong spread.

Using the known excitation and emission spectra of the Fusomer AgNCs in combination with the highly modular structure of fusomers allows for simple addition of targeting moieties. Furthermore, combined with the known excitation and emission spectra of Fusomer AgNCs, fusomers can be a tool for biosensing and bioimaging. Additionally, the fusomer and C12 hairpin AgNCs emit in the red-light range, which is the beginning of the range to penetrate human skin (42). Light at this wavelength can travel 4-5 mm underneath the surface of skin, further suggesting that Fusomer AgNCs could become internal biosensing molecules.

### REFERENCES

1. Chandler M, Panigaj M, Rolband L A, A fonin K A. Challenges to optimizing RNA nanostructures for large scale production and controlled therapeutic properties. Nanomedicine (Lond). 2020;15 (13): 1331-40.
2. Alvarez-Salas L M. Nucleic Acids as Therapeutic Agents. Curr Top Med Chem. 2008;8 (15): 1379-404.
3. Hong E, Halman J R, Shah A B, Khisamutdinov E F, Dobrovolskaia M A, Afonin K A. Structure and Composition Define Immunorecognition of Nucleic Acid Nanoparticles. Nano Lett. 2018;18 (7): 4309-21.
4. Hong E, Halman J R, Shah A, Cedrone E, Truong N, Afonin K A, et al. Toll-Like Receptor-Mediated Recognition of Nucleic Acid Nanoparticles (NANPs) in Human Primary Blood Cells. Molecules. 2019;24 (6).
5. Tran A N, Chandler M, Halman J, Beasock D, Fessler A, McKeough R Q, et al. Anhydrous Nucleic Acid Nanoparticles for Storage and Handling at Broad Range of Temperatures. Small. 2022;18 (13).
6. Rackley L, Stewart J M, Salotti J, Krokhotin A, Shah A, Halman J R, et al. RNA Fibers as Optimized Nanoscaffolds for siRNA Coordination and Reduced Immunological Recognition. Adv Funct M ater. 2018;28 (48).
7. Ke W, Hong E, Saito R F, Rangel M C, Wang J, Viard M, et al. RNA-DNA fibers and polygons with controlled immunorecognition activate RNAi, FRET and transcriptional regulation of NF-kappaB in human cells. Nucleic Acids Res. 2019;47 (3): 1350-61.
8. Halman J R, Satterwhite E, Roark B, Chandler M, Viard M, Ivanina A, et al. Functionally-interdependent shape-switching nanoparticles with controllable properties. Nucleic Acids Res. 2017;45 (4): 2210-20.
9. Johnson M, Halman J, Satterwhite E, Zakharov A, Bui M, Benkato K, et al. Programmable Nucleic Acid Based Polygons with Controlled Neuroimmunomodulatory Properties for Predictive QSAR Modeling. Small. 2017;13 (42).
10. Bui M, Johnson M, Viard M, Satterwhite E, Martins A, Li Z, et al. Versatile RNA tetra-U helix linking motif as a toolkit for nucleic acid nanotechnology. Nanomed-Nanotechnol. 2017;13 (3):1137-46.
11. Klotz K, Radwan Y, Chakrabarti K. Dissecting Functional Biological Interactions Using Modular RNA Nanoparticles. Molecules. 2023;28 (1).
12. Afonin K A, Lindsay B, Shapiro B A. Engineered RNA Nanodesigns for Applications in RNA Nanotechnology. DNA RNA Nanotechnol. 2015;1 (1): 1-15.
13. Paillart J C, Westhof E, Ehresmann C, Ehresmann B, Marquet R. Non-canonical interactions in a kissing loop complex: the dimerization initiation site of HIV-1 genomic RNA. J Mol Biol. 1997;270 (1): 36-49.
14. Shlyakhtenko L S, Gall A A, Filonov A, Cerovac Z, Lushnikov A, Lyubchenko Y L. Silatrane-based surface chemistry for immobilization of DNA, protein-DNA complexes and other biological materials. Ultramicroscopy. 2003;97 (1-4): 279-87.
15. Avila Y I, Chandler M, Cedrone E, Newton H S, Richardson M, Xu J, et al. Induction of Cytokines by Nucleic Acid Nanoparticles (NANPs) Depends on the Type of Delivery Carrier. Molecules. 2021;26 (3).
16. Gupta A K, Marshall N, Y ourston L, Rolband L, Beasock D, Danai L, et al. Optical, structural, and biological properties of silver nanoclusters formed within the loop of a C-12 hairpin sequence. Nanoscale Adv. 2023;5 (13): 3500-11.
17. Rolband L, Yourston L, Chandler M, Beasock D, Danai L, Kozlov S, et al. DNA-Templated Fluorescent Silver Nanoclusters Inhibit Bacterial Growth While Being Non-Toxic to Mammalian Cells. Molecules. 2021;26 (13).
18. Lyubchenko Y L, Gall A A, Shlyakhtenko L S. Visualization of DNA and Protein-DNA Complexes with Atomic Force Microscopy. In: Kuo J, editor. Electron Microscopy: Methods and Protocols. Totowa, NJ: Humana Press; 2014. p. 367-84.
19. Lushnikov A J, Avila Y I, A fonin K A, Krasnoslobodtsev A V. Characterization of RNA Nanoparticles and Their Dynamic Properties Using Atomic Force Microscopy. In: A fonin KA, editor. RNA Nanostructures: Design, Characterization, and Applications. New York, NY: Springer US; 2023. p. 191-202.
20. Sajja S, Chandler M, Fedorov D, Kasprzak W K, Lushnikov A, Viard M, et al. Dynamic Behavior of RNA Nanoparticles Analyzed by AFM on a Mica/Air Interface. Langmuir. 2018;34 (49): 15099-108.
21. Schindelin J, Arganda-Carreras I, Frise E, Kaynig V, Longair M, Pietzsch T, et al. Fiji: an open-source platform for biological-image analysis. Nat Methods. 2012;9 (7): 676-82.
22. Benkato K, O'Brien B, Bui M N, Jasinski D L, Guo P, Khisamutdinov E F. Evaluation of Thermal Stability of RNA Nanoparticles by Temperature Gradient Gel Electrophoresis (TGGE) in Native Condition. Methods Mol Biol. 2017;1632: 123-33.
23. Larkin J, Henley R Y, Muthukumar M, Rosenstein Jacob K, Wanunu M. High-Bandwidth Protein Analysis Using Solid-State Nanopores. Biophysical Journal. 2014;106 (3): 696-704.
24. Rosenstein J K, Wanunu M, Merchant C A, Drndic M, Shepard K L. Integrated nanopore sensing platform with sub-microsecond temporal resolution. Nature Methods. 2012;9 (5): 487-92.
25. Brewster J D. A simple micro-growth assay for enumerating bacteria. J Microbiol Meth. 2003;53 (1): 77-86.
26. Krasnoslobodtsev A V. Atomic Force Microscopy and Its Spectroscopic Combinations for Studying Self-Assembled Nanostructures. Supramolecular Nanotechnology2023. p. 823-44.
27. Adamcik J, Berquand A, Mezzenga R. Single-step direct measurement of amyloid fibrils stiffness by peak force quantitative nanomechanical atomic force microscopy. Applied Physics Letters. 2011;98 (19): 193701.
28. Adamcik J, Jung J-M, Flakowski J, De Los Rios P, Dietler G, Mezzenga R. Understanding amyloid aggregation by statistical analysis of atomic force microscopy images. Nature Nanotechnology. 2010;5 (6): 423-8.
29. Knowles T P J, Buehler M J. Nanomechanics of functional and pathological amyloid materials. Nature Nanotechnology. 2011;6 (8): 469-79.
30. Lamour G, Yip C K, Li H, Gsponer J. High Intrinsic Mechanical Flexibility of Mouse Prion Nanofibrils Revealed by Measurements of Axial and Radial Young's Moduli. ACS Nano. 2014;8 (4): 3851-61.
31. Schamberger B, Ziege R, Anselme K, Ben Amar M, Bykowski M, Castro A P G, et al. Curvature in Biological Systems: Its Quantification, Emergence, and Implications across the Scales. Advanced Materials. 2023;35 (13): 2206110.
32. Krasnoslobodtsev A V, Deckert-Gaudig T, Zhang Y, Deckert V, Lyubchenko Y L. Polymorphism of amyloid fibrils formed by a peptide from the yeast prion protein Sup35: A FM and Tip-Enhanced Raman Scattering studies. Ultramicroscopy. 2016;165:26-33.
33. Knowles T P, Fitzpatrick A W, Meehan S, Mott H R, Vendruscolo M, Dobson C M, et al. Role of Intermolecular Forces in Defining Material Properties of Protein Nanofibrils. Science. 2007;318 (5858): 1900-3.
34. Hadrien M, Gary J B. Kappa (&lt;em&gt;K&lt;/em&gt;): Analysis of Curvature in Biological Image Data using B-splines. bioR xiv. 2019:852772.
35. Tripathi P, Chandler M, Maffeo C M, Fallahi A, Makhamreh A, Halman J, et al. Discrimination of RNA fiber structures using solid-state nanopores. Nanoscale. 2022.
36. Alibakhshi M A, Halman J R, Wilson J, Aksimentiev A, Afonin K A, Wanunu M. Picomolar Fingerprinting of Nucleic Acid Nanoparticles Using Solid-State Nanopores. ACS Nano. 2017;11 (10): 9701-10.
37. Tripathi P, Chandler M, Maffeo C M, Fallahi A, Makhamreh A, Halman J, et al. Discrimination of RNA fiber structures using solid-state nanopores. Nanoscale. 2022;14 (18): 6866-75.
38. Jurrus E, Engel D, Star K, Monson K, Brandi J, Felberg L E, et al. Improvements to the APBS biomolecular solvation software suite. Protein Science. 2018;27 (1): 112-28.
39. Dolinsky T J, Czodrowski P, Li H, Nielsen J E, Jensen J H, Klebe G, et al. PDB 2PQR: expanding and upgrading automated preparation of biomolecular structures for molecular simulations. Nucleic Acids Research. 2007;35 (suppl_2): W 522-W5.
40. Porciani D, Tedeschi L, Marchetti L, Citti L, Piazza V, Beltram F, et al. A ptamer-Mediated Codelivery of Doxorubicin and NF-kappaB Decoy Enhances Chemosensitivity of Pancreatic Tumor Cells. M ol Ther Nucleic Acids. 2015;4 (4): e235.
41. Liu Z, Gong H, Zeng R, Liang X, Zhang L M, Y ang L, et al. Efficient delivery of NF-kappaB siRNA to human retinal pigment epithelial cells with hyperbranched cationic polysaccharide derivative-based nanoparticles. Int J Nanomedicine. 2015;10:2735-49.
42. Ash C, Dubec M, Donne K, Bashford T. Effect of wavelength and beam width on penetration in light-tissue interaction using computational methods. Lasers Med Sci. 2017;32 (8): 1909-18.
43. Roxo, C.; Kotkowiak, W.; Pasternak, A. G-Quadruplex-Forming Aptamers-Characteristics, Applications, and Perspectives. Molecules 625 2019, 24 (20), 3781.
44. Zavyalova, E.; Golovin, A.; Pavlova, G.; Kopylov, A. Module-Activity Relationship of G-quadruplex Based DNA A ptamers for Human Thrombin. Curr. Med. Chem. 2013, 20 (38), 4836-4843.
45. KeW, Chandler M, Cedrone E, Saito R F, Rangel M C, de Souza Junqueira M, Wang J, Shi D, Truong N, Richardson M, Rolband LA, Dreau D, Bedocs P, Chammas R, Dokholyan N V, Dobrovolskaia M A, Afonin K A. Locking and unlocking thrombin function using immunoquiescent nucleic acid nanoparticles with regulated retention in vivo. Nano Letters, 22 (14): 5961-5972, 2022. PM ID: 35786891

1. An RNA-DNA fusomer comprising a single-stranded polynucleotide comprising alternating RNA and DNA segments, wherein the polynucleotide self-assembles into a double-stranded DNA core with single-stranded RNA loops at either end of the fusomer.

2. The RNA-DNA fusomer of claim 1, wherein the polynucleotide comprises three DNA segments surrounding two RNA segments.

3. The RNA-DNA fusomer of claim 2, wherein the first and third DNA segments are complementary to the second DNA segment.

4. The RNA-DNA fusomer of claim 1, wherein one or more of the DNA segments comprise a therapeutic nucleic acid (TNA).

5. The RNA-DNA fusomer of claim 1, wherein the fusomer is capable of binding to a therapeutic agent, optionally wherein the therapeutic agent is a TNA.

6. (canceled)

7. The RNA-DNA fusomer of claim 1, wherein the DNA segments are between 4 and 30 nucleotides in length.

8. (canceled)

9. The RNA-DNA fusomer of claim 1, wherein the first and second RNA segments are between 8 and 20 nucleotides in length.

10. The RNA-DNA fusomer of claim 2, wherein the third DNA segment is longer than the first DNA segment.

11. The RNA-DNA fusomer of claim 10, wherein a portion of the third DNA segment is self-complementary, optionally wherein the self-complementary portion of the third DNA segment self-hybridizes to form a stem-loop structure.

12. (canceled)

13. The RNA-DNA fusomer of claim 11, wherein the stem-loop structure is capable of binding to a therapeutic agent, optionally wherein the therapeutic is an antimicrobial agent, optionally wherein the antimicrobial agent is a silver nanocluster (AgNC).

14-16. (canceled)

17. A composition comprising the RNA-DNA fusomer of claim 1.

18. A composition comprising two or more different RNA-DNA fusomers of claim 1, optionally wherein one RNA segment of one RNA-DNA fusomer hybridizes to one RNA segment of a second RNA-DNA fusomer and/or a third RNA-DNA fusomer, optionally wherein the hybridized RNA-DNA fusomers assemble into one or more nucleic acid fibers.

19-20. (canceled)

21. A method of for modulating expression of a target nucleic acid molecule and/or protein in a cell, the method comprising contacting the cell with the RNA-DNA fusomer of claim 1, wherein the cell expresses the nucleic acid and/or protein targeted by the RNA-DNA fusomer.

22. A method for deactivating a protein (e.g., an enzyme) in a cell, the method comprising contacting the cell with the RNA-DNA fusomer of claim 1, wherein the RNA-DNA fusomer binds to the protein and deactivates it.

23. A method for inhibiting the growth of a microorganism, the method comprising contacting the microorganism with the RNA-DNA fusomer of claim 1, thereby inhibiting the growth of the microorganism.

24. A method for reducing blood coagulation in a subject, the method comprising administering to the subject a therapeutically effective amount of the RNA-DNA fusomer of claim 1,thereby reducing blood coagulation.

25. A method for modulating an immune response in a subject, the method comprising administering to the subject a therapeutically effective amount of the RNA-DNA fusomer of claim 1, thereby modulating the immune response in the subject.

26. A method for modulating an inflammatory response in a subject, the method comprising administering to the subject a therapeutically effective amount of the RNA-DNA fusomer of claim 1, thereby modulating the inflammatory response in the subject.

27. A method for detecting a target protein by a biosensing nanopore, the method comprising contacting the target protein with the RNA-DNA fusomer of claim 1 and determining the capture rate of the RNA-DNA fusomer by the biosensing nanopore, thereby detecting the target protein by the biosensing nanopore.

28. (canceled)

29. A method for delivering a therapeutic agent to a subject, the method comprising contacting the RNA-DNA fusomer of claim 1 with the therapeutic agent, wherein the RNA-DNA fusomer binds to the therapeutic agent, and then administering the RNA-DNA fusomer that is bound to the therapeutic agent to the subject, thereby delivering the therapeutic agent to the subject.

30. (canceled)
